(19) United States
(12) Patent Application Publication
(10) Pub. No.: US 2017/0198307 A1
(43) Pub. Date: Jul. 13, 2017

(54) METHODS FOR TARGETED MODIFICATION OF GENOMIC DNA

(71) Applicants: PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Gerald MARSISCHKY, Newton, MA (US); Bradley L. PENTELUTE, Cambridge, MA (US); R. John Collier, Wellesley, MA (US); Andrew J. MCCLUSKEY, Shrewsbury, MA (US)

(73) Assignees: PRESIDENT AND FELLOWS OF HARVARD COLLEGE, Cambridge, MA (US); MASSACHUSETTS INSTITUTE OF TECHNOLOGY, Cambridge, MA (US)

(21) Appl. No.: 15/316,218

(22) PCT Filed: Jun. 5, 2015
(86) PCT No.: PCT/US15/34471

§ 371 Date: Dec. 5, 2016

Related U.S. Application Data
(60) Provisional application No. 62/008,547, filed on Jun. 6, 2014.

(51) Int. Cl. C12N 15/90; C12N 9/54; C07K 14/195; C12N 9/22

(52) U.S. Cl. CPC C12N 15/907; C12N 9/22; C12N 9/54; C12Y 304/24083; C12Y 301/21004; C07K 14/195; C07K 2319/70; C07K 2319/09; C07K 2319/81; C07K 2319/80

(57) ABSTRACT

Provided herein are methods of integrating one or more exogenous nucleic acids into one or more selected target sites of a host cell genome. In certain embodiments, the methods comprise contacting the host cell genome with one or more nucleic acid constructs fused with a pore-forming toxin, which may further be targeted to a particular cell type, wherein the nucleic acid constructs comprise an exogenous nucleic acid to be integrated into a genomic target site, and an enzyme, such as a nuclease or recombinase or a combination thereof capable of targeted introduction of the nucleic acid into the genome.

### CROSS-REFERENCE TO RELATED APPLICATIONS

This application claims benefit under 35 U.S.C. §119(e) of U.S. Provisional Application No. 62/008,547 filed Jun. 6, 2014, the contents of which are incorporated herein by reference in their entirety.

### GOVERNMENT SUPPORT

This invention was made with government support under contract No. AI22021 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 3, 2015, is named 002806-078051-PCT_SL.txt and is 3,081 bytes in size.

### TECHNICAL FIELD

The present invention relates to the field of genetic engineering methods.

### BACKGROUND

Targeted genetic modification is important for any practical genome engineering application.

Artificial nucleases, such as zinc finger nucleases (fusions of zinc finger domains and cleavage domains) for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, United States Patent Publications 20030232410; 20050208489; 20050026157; 20050064474; 20060188987; 20060063231; and International Publication WO 07/014,275.

To increase specificity, a pair of fusion proteins, each comprising a zinc finger binding domain and cleavage half-domain can be used to cleave the target genomic DNA. Because cleavage does not occur unless the cleavage half-domains associate to form a functional dimer, this arrangement increases specificity.

To further decrease off-target cleavage events, engineered cleavage half-domains, for example domains that form obligate heterodimers, have also been developed. See, e.g., U.S. Patent Publication No. 2008/0131963.

Typically, TALENs and ZFNs are delivered to cells via transfected expression vectors, a method with obvious long-term shortcomings when creating engineered cells. Additionally, transfection often results in cytotoxicity due to the prolonged duration and high level of expression involved. The current most practical TALEN and ZFN delivery methods include microinjection of cells with RNA transcripts encoding TALEN proteins. While it is a low throughput method, RNA microinjection has been used to create gene knockouts with high efficiency in cells without the use of drug selection. In another method, ZFN proteins tagged with a TAT-like domain have been shown to be capable of knocking out genes when added directly to mammalian cells. The uptake of these proteins into cells does not appear to be dependent on a cell receptor.

However, modifying nuclear genomes or delivering genetic material into a nucleus of a cell or a population of target cells is often either very inefficient or results in adding residual genetic material into the cell in the form of a vector, such as a viral vector, e.g., lentiviral, retroviral or adenoviral vector. Moreover, most of the genetic engineering techniques lack means to target a particular cell or cell population in a mixed cell population, such as a tissue or an organ.

Therefore, additional safe and efficient techniques for site-specific genome modification are needed.

### SUMMARY OF THE INVENTION

We describe a novel approach to address targeted genome modification applications by describing compositions and methods for delivery of large or complex biomolecules, such as genome modifying enzymes into cell, or cell compartments, such as into cellular nuclei or mitochondria, utilizing pore forming toxins.

The invention is based, at least in part of our surprising discovery, that bacterial toxin pores can deliver large cargo of external material, such as entire enzymes, into cells, and that those cargoes can be further directed to an intracellular compartment, such as the nucleus or mitochondria. Moreover, we have found that the delivered enzymes retain their function and thus the system can be surprisingly utilized for targeted genome editing applications.

For example, we describe components for a system and the system that utilizes bacterial toxin pores to deliver engineered site-specific enzymes, such as topoisomerase, nuclease or recombinase proteins into mammalian cells and nuclei and methods of making directed changes in genomic DNA using such engineered site-specific enzymes.

We further describe how the enzymes can be targeted to a specific cell using, e.g., ligands, antibodies or affybodies fused to a pore-forming toxin cellular targeting peptide so that the pores will form only to specific cells thus facilitating cell-type specific targeting. The biomolecule, such as the enzyme, can be further engineered to comprise an intracellular localization signal allowing its delivery to a specific intracellular location, such as a nucleus.

The compositions and methods described herein allow site-specific genome modification either in the nucleus or in mitochondria without adding a genomic imprint of the delivery system.

The compositions and methods described herein provide an ideal system for targeting cells both in vitro and in vivo, such as targeting particular neurons in the brains for site-specific genetic manipulation or targeting specific cells in circulating blood in vivo or ex vivo for the same.

We have previously developed a system capable of efficiently translocating heterologous small proteins, e.g., DTA, the catalytic domain of diphtheria toxin, into cells using components of the anthrax lethal toxin: the protective antigen protein (PA) which contains the host cell receptor binding domain and forms a heptameric pore in the acidified endosome, and a translocation tag (LFn) derived from the amino terminus of the lethal factor protein. This system has also been extended to allow targeted translocation of LFn-tagged proteins into cells via a variety of cell surface proteins. However, the previous uses of this system have concentrated on delivery of nucleic acids, short peptides and protein fragments, such as epitopes (U.S. Pub. 2003-0202989), modified nucleic acids (PCT/US2013/027307) and small molecules (WO2013177231).

We have now discovered that this system can surprisingly deliver very large “payloads” or cargo, such as entire enzymes, e.g., genome-modifying enzymes into the cell and specifically, into the nucleus of a cell. Moreover, we surprisingly discovered that such enzymes retain their activity after having been transported through the pore-forming toxin units.

Thus, we provide compositions comprising engineered genome modifying enzymes and novel methods in which toxin protein translocation systems are used to directly introduce enzymes, such as topoisomerases, nucleases and recombinases, into cells and cellular nuclei to facilitate targeted modification of genomes.

For example, we have shown that TALEN proteins can be introduced to a cell to knockout of target genes. In this proof-of-concept experiment, we introduced purified LFn-tagged TALEN proteins into cells via anthrax protective antigen (PA) pores to allow specific cuts at a genomic DNA target sequence followed by non-homologous end-joining DNA repair and loss of gene function though reading frame alterations (FIG. 1).

Accordingly, we describe compositions comprising an engineered large biomolecule, e.g., a genome-modifying enzyme fused with a pore-specific delivery protein or tag. In some aspects, such compositions further comprise an intracellular localization signal, such as nuclear or mitochondrial localization signal.

We also describe methods of modifying genomes comprising contacting the living cell with a pore forming protein and one or more fusion molecules comprising a pore specific delivery protein linked to a genome-modifying enzyme, wherein the genome modifying enzyme is delivered to the nucleus of the living cell in an effective amount for performing its target modification, i.e., genome modification. In some aspects of all the embodiments of the invention, the method further comprises contacting the cell with a nucleic acid, such as a gene encoding a protein desired to be delivered to the cell. The gene can be delivered using a vector, such as a viral vector, e.g., adenoviral, retroviral or lentiviral vectors or using a fusion construct, comprising a pore specific delivery protein linked to the nucleic acid as described, e.g., in U.S. Pub. 2003-0202989.

The methods may be further enhanced by targeting the pore-forming protein to a cell expressing a particular receptor or ligand or epitope as described in PCT/US2013/027307. Accordingly, the methods of the invention also allow targeting a cell or a population of cells in a tissue or organ, with a site-specific genome modification.

Accordingly, some aspects of the invention provide an engineered site-specific nuclease protein comprising a site-specific nuclease protein and a protein tag capable of binding to a pore forming protein. In some or all aspects of the invention, the engineered site-specific nuclease protein further comprises a nuclear localization signal. In some or all aspects of the invention, the engineered site-specific nuclease protein is selected from a zinc-finger nuclease, a transcription activator-like effector nuclease, and a monomeric site-specific nuclease.

In some or all aspects of the invention, the monomeric site-specific nuclease can be an engineered GIY-YIG family protein.

In some or all aspects of the invention, the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.

In some aspects, the invention provides a method for delivering an engineered site-specific nuclease into a mammalian cell comprising contacting the mammalian cell with a pore forming protein and the engineered site-specific nuclease as described herein. In some or all aspects of the invention, the pore forming protein further comprises a cell-targeting peptide.

In some or all aspects of the invention, the cell-targeting peptide is selected from a cell receptor ligand, an antibody, or an affibody.

In some aspects, the invention provides a method for site-specific modification of the genome of a mammalian cell comprising the steps of contacting the mammalian cell with a pore forming protein and the engineered site-specific nuclease as described herein. In some or all aspects of the invention, the core forming protein further comprises a cell targeting peptide. In some or all aspects of the invention, the cell-targeting peptide is selected from a cell receptor ligand, an antibody, or an affibody.

In some or all aspects of the invention, the mammalian cell is contacted with at least two pairs of the engineered site-specific nucleases as described herein, wherein each of the at least two pairs of the engineered site-specific nucleases target a different receptor and are active only when transported via the different receptors into the same mammalian cell, thus minimizing both off-target activity and activity in non-target cells.

In some or all aspects of the invention, the off-target activity comprises cutting DNA at a single TALEN binding site. In some or all aspects of the invention, the at least two pairs of the engineered site specific nucleases comprise a TALEN and a CRISPR nuclease targeted to adjacent sequences in which Fold nuclease dimerization is required for cutting.

In some or all aspects of the invention, the method comprises use of a nicking version of the FokI nuclease in TALE or Cas9 fusions targeted to adjacent sequences, wherein the targeted nucleases bind and nick DNA separately, and a double strand break forms if the second cut was made before the first was repaired.

In some or all aspects of the invention, the method further comprises protein complementation, wherein at least one of the nucleases is divided into two separate and inactive domains which are fused to TALE or Cas9 proteins, wherein DNA double strand breaks occur only when the fusions bind to their target sequences in the correct orientation.

In some or all aspects of the invention, the method further comprises a step of contacting the cell with a replacement nucleic acid along with the enzyme component.

In some or all aspects of the methods of the invention, the mammalian cell is a neuronal system cell.

In some or all aspects of the methods of the invention, the method is performed in vitro.

In some or all aspects of the methods of the invention, the method is performed in vivo.

In some or all aspects of the methods of the invention, the mammalian cell is a human cell.

In some aspects, the invention provides an engineered site-specific recombinase protein comprising a site-specific recombinase protein linked to protein tag capable of binding to a pore forming protein. In some or all aspects of the invention, the engineered site-specific recombinase protein further comprises a nuclear localization signal.

In some or all aspects of the invention, the site-specific recombinase is a tyrosine recombinase or a serine recombinase. In some or all aspects of the invention, the tyrosine recombinase is Flp-recombinase or Cre-recombinase. In some or all aspects of the invention, the serine recombinase is PhiC31 Integrase.

The phrase “desired sequence” is used herein to refer to a sequence in a genome, wherein the engineering is meant to target. Engineered recombinases such as TALE-recombinase or catalytically dead Cas9 allow targeting of any sequence, not just loxP or FRT sites, as a target sequence can be “programmed” into the system.

In some or all aspects of the invention, the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.

In some aspects, the invention provides a method for delivering an engineered site-specific recombinase into a mammalian cell comprising contacting the mammalian cell with a pore forming protein and the engineered site-specific recombinase as described in any aspect of the invention. In some or all aspects of the invention, the pore forming protein further comprises a cell-targeting peptide. In some or all aspects of the invention, the cell-targeting peptide is selected from a cell receptor ligand, an antibody, or an affibody.

In some aspects, the invention also provides a method for site-specific modification of the genome of a mammalian cell comprising the steps of contacting the mammalian cell with a pore forming protein and the engineered site-specific recombinase as described herein. In some or all aspects of the invention, the core forming protein further comprises a cell targeting peptide.

In some or all aspects of the invention, the cell-targeting peptide is selected from a cell receptor ligand, an antibody, or an affibody. In some or all aspects of the invention, the mammalian cell is contacted with at least two pairs of the engineered site-specific recombinases as described herein, wherein each of the at least two pairs of the engineered site-specific recombinases target a different receptor and are active only when transported via the different receptors into the same mammalian cell, thus minimizing both off-target activity and activity in non-target cells. In some or all aspects of the invention, the off-target activity comprised cutting DNA at a single recombinase binding site.

In some or all aspects of the invention, the methods further comprise a step of contacting the cell with a replacement nucleic acid.

In some or all aspects of the invention, the mammalian cell is a neuronal system cell.

In some or all aspects of the invention, the method is performed in vitro.

In some or all aspects of the invention, the method is performed in vivo.

In some or all aspects of the invention, the mammalian cell is a human cell.

In some aspects, the invention provides an engineered site-specific enzyme protein comprising a site-specific enzyme protein linked to protein tag capable of binding to a pore forming protein. In some or all aspects of the invention, the engineered site-specific enzyme protein further comprises a nuclear localization signal.

In some or all aspects of the invention, the site-specific enzyme protein is a LAGLIDADG homing endonuclease (“LAGLIDADG” disclosed as SEQ ID NO: 1).

In some or all aspects of the invention, the endonuclease is a monomeric endonuclease.

In some or all aspects of the invention, the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.

In some aspects, the invention provides s method for delivering an engineered site-specific enzyme protein into a mammalian cell comprising contacting the mammalian cell with a pore forming protein and the engineered site-specific enzyme protein as described herein.

In some or all aspects of the invention, the pore forming protein further comprises a cell-targeting peptide.

In some or all aspects of the invention, the cell-targeting peptide is selected from a cell receptor ligand, an antibody, or an affibody.

In yet some aspects, the invention provides a method for site-specific modification of the genome of a mammalian cell comprising the steps of contacting the mammalian cell with a pore forming protein and the engineered site-specific enzyme protein as described herein.

In some or all aspects of the invention, the pore forming protein further comprises a cell targeting peptide.

In some or all aspects of the invention, the cell-targeting peptide is selected from a cell receptor ligand, an antibody, or an affibody.

In some or all aspects of the invention, the mammalian cell is contacted with at least two pairs of the engineered site-specific nucleases of any one of the 43-50, wherein each of the at least two pairs of the engineered site-specific nucleases target a different receptor and are active only when transported via the different receptors into the same mammalian cell, thus minimizing both off-target activity and activity in non-target cells.

In some or all aspects of the invention, the off-target activity comprised cutting DNA at a single enzymatic cleavage site.

In some or all aspects of the invention, the method further comprises a step of contacting the cell with a replacement nucleic acid.

In some or all aspects of the invention, the mammalian cell is a neuronal system cell.

In some or all aspects of the invention, the method is performed in vitro.

In some or all aspects of the invention, the method is performed in vivo.

In some or all aspects of the invention, the mammalian cell is a human cell.

### BRIEF DESCRIPTION OF DRAWINGS

FIGS. 1A-1C depict an example of the method, where purified Antrax toxin lethal factor n-terminal (LFn)-tagged TALEN proteins were introduced into cells via protective antigen pores to allow specific cuts at a genomic DNA target sequence followed by non-homologous end joining DNA repair and loss of gene function though reading frame alterations. FIG. 1A depicts a schematic of a LFn-tagged TALEN protein. Figure discloses SEQ ID NO: 6. FIG. 1B depicts a schematic of the uptake of TALENS proteins into the cell. FIG. 1C depicts schematic of the alteration of the genomic DNA.

FIGS. 2A-2C demonstrate that both purified and partially purified LFn-TALEN proteins have site-specific endonuclease activity in vitro. FIG. 2A depicts a schematic of a LFn-TALEN protein. Figure discloses SEQ ID NOS 7 and 8, respectively, in order of appearance. FIG. 2B depicts the purification of the LFn-TALEN proteins. FIG. 2C depicts a TALEN nuclease assay.

FIG. 3 demonstrates that both purified and partially purified LFn-TALEN proteins can induce loss of function in a counter-selectable gene (DPH1) when added to human cells along with protective antigen.

FIG. 4 depicts the LCMS trace of purified single zinc finger fused to LFN-DTA by sortagging.

FIG. 5 demonstrates LFN-DTA-ZF can be delivered as effectively as the positive control LFN-DTA by protein synthesis inhibition assay.

FIG. 6 depicts the delivery of LFN-DTA-ZF by western blot. The LFN-DTA-ZF band can only be observed in the absence of wild type PA. In the presence of PA(F427H), a PA mutant that abolishes its translocation ability, no band can be observed.

FIG. 7 depicts the LCMS trace of purified LFN-DTA-FokI.

FIG. 8 demonstrates that LFN-DTA-FokI has a two-log shift compared to LFN-DTA, indicating translocation is affected by FokI most likely due to the presence of cysteine.

FIG. 9 demonstrates the translocation of LFN-DTA-FokI by western blot in HEK293T cells. The LFN-DTA-FokI band can still be seen in the presence of 200 nM of LFN-DTA-FokI and 40 nM of wild-type PA after 24 hours of incubation despite its affected translocation efficiency, implying the some amount of LFN-DTA-FokI can still be delivered into cells by PA.

FIG. 10 depicts the experimental strategy for delivery of LFN-ZFN.

FIG. 11 depicts LFN-ZFNR purification by Ni column.

FIG. 12 depicts LFN-ZFNL purification by Ni column.

FIG. 13 depicts the results of an in vitro cleavage assay performed using the combination of LFN-ZFNR and LFN-ZRNL, demonstrating that the combination of proteins has cleavage activity, indicated by upward shifts of the bands.

FIGS. 14-16 depict the purification of LFN-ZFNR/L.

FIG. 17 depicts the activity of LFN-ZFNR/L in BT474 cells measured by surveyor nuclease assays.

FIGS. 18-23 depict purification optimization of LFN-ZFNR.

FIG. 24 depicts a western blot of BT474 cells treated with LFN-ZFNR.

FIG. 25 depicts a western blot of BT474 cells treated with LFN-ZFNL.

FIG. 26 depicts the results of a surveyor nuclease assay for HEK cells transfected with ZFN or LFN-ZFN.

FIG. 27 depicts the effect of multiple treatments and serum. BT474 cells were subjected to single or three treatments of LFNR/L and PA in the presence or absence of serum.

### DETAILED DESCRIPTION OF THE INVENTION

We describe novel compositions and methods for introducing large functional biomolecules, such as enzymes, such as enzymes capable of facilitating targeted genetic changes into cells, such as natural and artificial living cells. The methods also allow delivery of the nucleic acids in combination with the targeting enzymes, such as nucleases and recombinases into a cell, e.g., into the nucleus of the cell. The methods leave no genetic trace of the delivery method in the cell. Moreover, the methods allow delivery to targeted cells or cell populations in vivo or in vitro by using a targeted delivery vehicle comprising a toxin pore-forming protein.

At its most basic, the invention is platform to deliver a functional biomolecule or complex or large biomolecule, such as an enzyme, for example, a molecule of at least 40 kDa, 50 kDa, 60 kDa, 70 kDa, 80 kDa, 90 kDa or more, at least 100 kDa, at least 100 kDa to 500 kDa, at least 110 kDa, 120 kDa, 130 kDa, 140 kDa, 150 kDa, or more, such as at least 200 kDa biomolecule in size into the cell, including into an intracellular compartment of the cell, such as nucleus. The delivery platform is based on a non-toxic form of a toxin protein, referred to as a delivery peptide or a pore specific delivery protein that specifically interacts with a cognate pore forming protein to achieve specific cellular internalization. Cargos to be delivered to a living cell may be covalently linked to the delivery peptide using, e.g., a transpeptidase sortase A or other chemical reaction. Once delivered to the cell that has been loaded with pore forming protein the cargo and delivery peptide are transported to the cell cytosol in the presence of protective antigen, and can be further delivered to an intracellular location using a further localization peptide, fused to the complex biomolecule, such as an enzyme, like genome modifying enzyme.

Specifically, the invention provides genome-editing methods and systems for delivery of genome-editing enzymes into cells using targeted cellular or nuclear delivery with bacterial toxins.

The compositions and methods of the invention comprise a complex biological molecule. In some aspects, the complex biological molecules are complete functional proteins, such as enzymes. Enzymes according to the present invention typically include enzymes that can modify genomic DNA, including topoisomerases, such as topoisomerase I and II, nucleases, recombinases, invertases, and integrases. The term “genome modifying enzyme” as used herein encompasses any enzyme capable of modifying genomic nucleic acids, such as DNA or RNA. For example, topoisomerases, meganucleases, zinc-finger nucleases (ZFNs), transcription activator-like effector nucleases (TALENs), and site-specific recombinases (SSRs) can all be combined in highly efficient “tag-and-exchange technologies” (Branda, Catherine S.; Dymecki, Susan M. (2004). Developmental Cell 6 (1): 7-28), which can all be potentiated or enhanced using the compositions and methods described herein. Examples of enzymes also include tyrosine-recombinases, such as Cre, Dre, Flp, KD, B2 B3 and serine-integrases.

An example of a genome modifying enzyme is an artificially created transcription activator-like effector nucleases (TALENs) are artificial restriction enzymes generated by fusing a TAL effector DNA binding domain to a DNA cleavage domain. Transcription activator-like effectors (TALEs) can be quickly engineered to bind practically any desired DNA sequence (Boch, Jens (February 2011). Nature Biotechnology 29 (2): 135-6). By combining such an engineered TALE with a DNA cleavage domain, one can engineer restriction enzymes that are specific for any desired DNA sequence.

Examples of other genome modifying enzymes include Cre (“causes recombination”) which is able to recombine specific sequences of DNA without the need for cofactors. The enzyme recognizes 34 base pair DNA sequences called loxP (“locus of crossover in phage P1”).

Another example is Flp. In its natural host (S. cerevisiae) the Flp/FRT system enables replication of a “2μ plasmid” by the inversion of a segment that is flanked by two identical, but oppositely oriented FRT sites (“flippase” activity). This inversion changes the relative orientation of replication forks within the plasmid enabling “rolling circle”—amplification of the circular 2μ entity before the multimeric intermediates are resolved to release multiple monomeric products. Whereas 34 bp minimal FRT sites favor excision/resolution to a similar extent as the analogue loxP sites for Cre, the natural, more extended 48 bp FRT variants enable a higher degree of integration, while overcoming certain promiscuous interactions as described for phage enzymes like Cre- (Nern, A.; et al. (2011) Proceedings of the National Academy of Sciences 108 (34): 14198) and PhiC31 (Turan, S.; Bode, J. (2011)The FASEB Journal 25 (12): 4088-107).

Yet another example is PhiC31 Integrase. Contrary to the Tyr recombinases, such as Cre and Flp, PhiC31-INT as such acts in a unidirectional manner, locking in the donor vector at a genomically anchored target. An obvious advantage of this system is that it can rely on unmodified, native attP (acceptor) and attB donor sites. Additional benefits, together with certain complications, arise from the fact that mouse and human genomes per se contain a limited number of endogenous targets, so called “attP-pseudosites.”

A further example is I-AniI Nickase (I-AniI), encoded by a group I intron harbored within the Aspergillus nidulans apocytochrome B oxidase gene, cleaves a 19-bp asymmetric DNA target (McConnel Smith A, et al., PNAS vol. 106 no. 13, 5099-5104).

Recombinase-mediated cassette exchange (RMCE) is also a useful technique for introducing multiple alleles or markers efficiently into a defined site in the genome. It is accomplished by surrounding the cassette DNA to be exchanged with recombination sites (e.g., loxP) in both the targeting and the genomic target sequences in cells expressing the relevant recombinase (e.g., Cre).

A version of RMCE has been developed that utilizes two recombinase systems, Cre/loxP and Flp/FRT, to mediate the fragment exchange in way that mitigates the reversibility inherent to these recombination systems (Lauth et al., Nucleic Acids Research, 2002, Vol. 30 No. 21 e115). In this version of RMCE, the cassette DNA in both the donor (targeting) and genomic sites is flanked by LoxP and FRT sites (i.e., as loxP-cassette-FRT), and the recombination takes place in cells expressing both the Flp and Cre recombinases. An improved version of the method utilizes balanced co-expression of Cre and Flp recombinases, that are expressed from a single promoter as Flp-2A-Cre or Flp-IRES-Cre, and yields a high fraction of cells with fragment replacement (Anderson et al., Nucleic Acids Research, 2012, Vol. 40, No. 8 e62).

One can also solve the reversibility using another common approach to the Cre and Flp recombinase, namely using variant recombination sites at either end of the DNA to be exchanged (e.g., the loxP site variants used in CREATOR™ (Clontech) or Univector cloning (see, e.g. Liu et al. Curr Biol. 1998 Dec. 3; 8(24):1300-9) and the att site variants used in GATEWAY® cloning (Invitrogen).

One can also use RMCE using dual transposon integrases, e.g., piggybac or Sleeping Beauty, or for a dual integrase, such as PhiC31 Integrase and a partner, version of integrase-mediated site-specific insertion (IMSI).

For example, one can prepare constructs such as TALE-recombinases (TALERs, Nucleic Acids Res. 2012 November;40(21):11163-72) or fusions of catalytically dead Cas9 to recombinases rather than to site-specific recombinases like Cre or Flp. Sequence-progammable recombinases can in some instances be more useful than site-specific recombinases since any sequence can be targeted, and not just a loxP or FRT site. The phrase “desired sequence” is used herein to refer to a sequence in a genome, wherein the engineering is meant to target. Engineered recombinases such as TALE-recombinase or catalytically dead Cas9 allow targeting of any sequence, not just loxP or FRT sites, as a target sequence can be “programmed” into the system.

Catalytically inactive or ‘dead’ Cas9 (dCas9) can be created, e.g., with mutations in both the RuvC and HNH domains. This can be recruited by a gRNA without cleaving the target DNA site. Catalytically inactive dCas9 can be fused to a heterologous effector domain.

In some embodiments, the genome modifying enzyme comprises a DNA binding domain that is an engineered domain from a TAL effector derived from the plant pathogen Xanthomonas (see, Miller et al. (2010) Nature Biotechnology, December 22 [Epub ahead of print]; Boch et al, (2009) Science 29 Oct. 2009 (10.1126/science.117881) and Moscou and Bogdanove, (2009) Science 29 Oct. 2009 (10.1126/science.1178817); see, also, U.S. Publication No. 20110301073, the disclosures of which is hereby incorporated by reference in its entirety. In some embodiments, the TALE DNA binding domain is fused to a FokI cleavage as described, resulting in a TALE-nuclease (TALEN).

In some aspects of the invention, complex biological molecule does not include a non-naturally occurring or naturally occurring small molecules, such as small molecules of less than 900 Da, including epitopes, natural or non-natural peptides or protein fragments of under 25 kDa, or modified or unmodified nucleic acids.

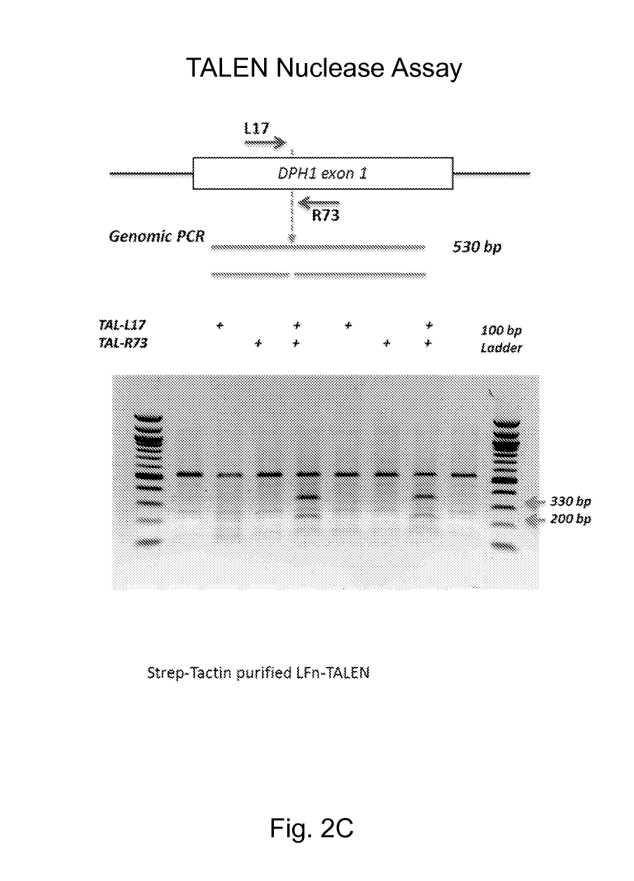

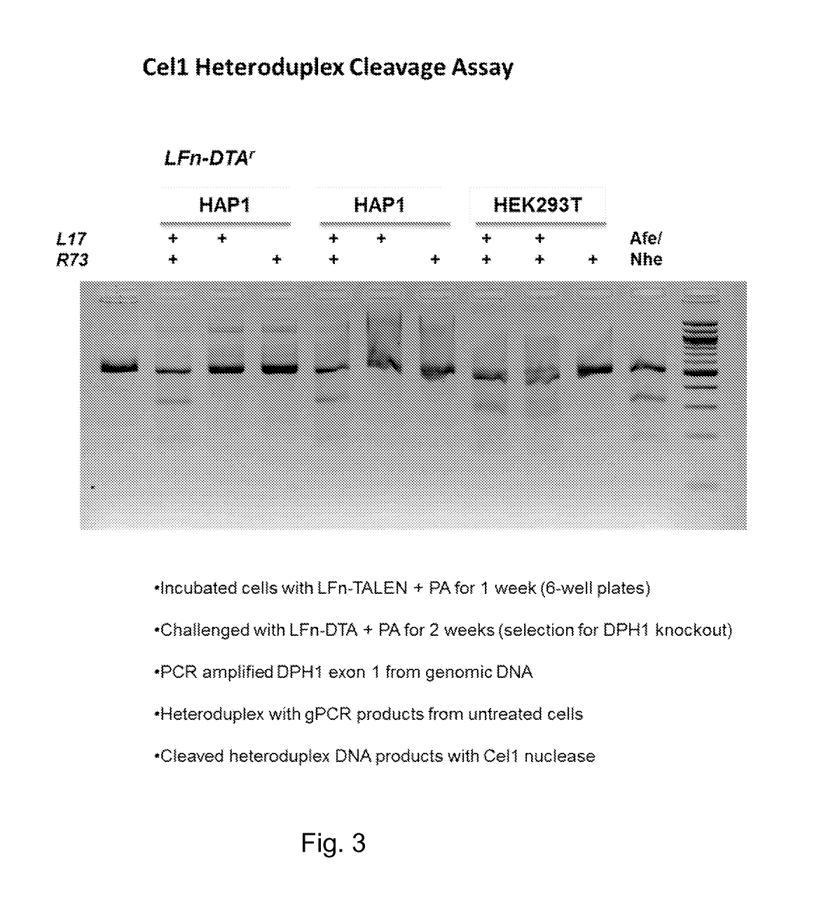

For example, we have shown that purified and partially purified LFn-TALEN proteins have site-specific endonuclease activity in vitro (FIG. 2), and induce loss of function in a counter-selectable gene (DPH1) when added to human cells along with protective antigen (FIG. 3). Heteroduplex/nuclease assays have confirmed that the target site in the DPH1 gene was altered in the treated cells. This experiment shows that the genome modifying nuclease is not only introduced to the nuclear location but also retains its functionality.

The functional delivery of LFn-TALEN proteins via pore-forming toxin, such as PA is a novel means of using TALENs for genetic manipulation that leaves no transfected DNA “footprint” in the altered genome. This is a large advantage over methods using viral vectors. Moreover, the methods are much more efficient than microinjection, wherein each cell must be manipulated individually.

Moreover, the pore forming toxin, such as the PA-mediated delivery system that we used in our example, has other important advantages over other enzyme delivery systems, such as the currently known TALEN and ZFN delivery systems. For instance, the receptor binding specificity of protective antigen can be reprogrammed to enable the delivery of toxin proteins to specific target cells. For example, LFn-tagged enzyme, such as TALENs or ZFNs targeted in this way are useful in creating genome modifications in a specific cell type within mixed cell populations, such as organs, and in whole animals. Target-cell specificity may also be further enhanced by the utilization of different cell receptors for delivery of each of the pore forming toxin targeting delivery proteins, such as LFn-TALEN proteins.

In addition, many of the genome modifying enzymes, such as TALENs and ZFNs, have been demonstrated to have off-target activity in genomic DNA. With the current method, it is possible to suppress off-target cutting, for example, by translocating low concentrations of LFn-TALENs via different cell receptors to minimize the use of secondary binding sites in the genome. Off-target cutting can also be addressed with the use of conditional nucleases, for instance by creating TALEN-fusions built with fragments of the Staphylococcus aureus nuclease. Such TALEN fusions exhibit nuclease activity only when protein complementation occurs between the nuclease fragments at properly oriented adjacent TALE binding sites in DNA. Furthermore, as an endo/exonuclease it creates a deletion large enough to minimize the restoration of functional coding sequence by DNA repair at the cut site.

Gene replacement can also be facilitated by the described targeted enzymes, such as LFn-TALEN delivery system, provided that two pairs of targeted enzymes, such as LFn-TALENs were introduced to create double-strand breaks on either side of the genomic DNA target site. The DNA used for the replacement can be added as usual either by transfection or by a viral delivery system.

We have shown that an enzyme with the size of TALEN can be translocated to nucleus with a LFn-tag. Thus, the LFn-tag/protective antigen delivery system can be applied to any DNA-modifying enzymes such as site-specific recombinases, or to the site-specific targeting of enzymes for modifying gene expression through local changes in chromosomal epigenetic coding, e.g., with an LFn-TALE-histone deacetylase fusion protein.

Similarly, versions of these reagents might also be useful as specific biochemical probes in proteomics experiments. For instance, a fusion protein can be made of a DNA-binding domain, such as an LFn-TALE protein, with a DNA topoisomerase mutant domain that accumulates a dead-end product of the normal topoisomerase/DNA thioester intermediate.

This covalent adduct provides a useful affinity purification handle for the target DNA and enables, e.g., Reverse-ChIP experiments for the identification of proteins bound nearby the TALE target sequence.

Accordingly, the system can be used utilizing multiple different toxin delivery systems for translocating large proteins, such as enzymes, such as genome modifying enzymes, e.g., TALENs, into cells.

The term “engineered” used herein and throughout the specification in the context of engineered enzymes or engineered genome modifying enzymes means that the known enzyme has been modified to include a protein tag that directs the enzyme to a toxin pore forming protein. The tags are dependent on which pore-forming toxin is used to deliver the enzyme into a cell that has been targeted by the pore-forming protein and can be selected by a skilled artisan based on the used toxin delivery system. The term “engineered” means that the tag has been added to the enzyme either as a protein or the enzyme has been recombinantly produced to include the desired pore forming toxin recognizing tag.

It is well known that the Clustered Regularly Interspaced Short Palindromic Repeats (CRISPR) Type II system is currently the most commonly used RNA-Guided Endonuclease technology for genome engineering. There are two distinct components to this system: (1) a guide RNA and (2) an endonuclease, in this case the CRISPR associated (Cas) nuclease, Cas9. The guide RNA is a combination of the endogenous bacterial crRNA and tracrRNA into a single chimeric guide RNA (gRNA) transcript. The gRNA combines the targeting specificity of the crRNA with the scaffolding properties of the tracrRNA into a single transcript. When the gRNA and the Cas9 are expressed in the cell, the genomic target sequence can be modified or permanently disrupted.

The gRNA/Cas9 complex is recruited to the target sequence by the base-pairing between the gRNA sequence and the complement to the target sequence in the genomic DNA. For successful binding of Cas9, the genomic target sequence must also contain the correct Protospacer Adjacent Motiff (PAM) sequence immediately following the target sequence (learn more about PAM sequences). The binding of the gRNA/Cas9 complex localizes the Cas9 to the genomic target sequence so that the wild-type Cas9 can cut both strands of DNA causing a Double Strand Break (DSB). A DSB can be repaired through one of two general repair pathways: (1) the Non-Homologous End Joining (NHEJ) DNA repair pathway or (2) the Homology Directed Repair (HDR) pathway. The NHEJ repair pathway often results in inserts/deletions (InDels) at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template.

We describe here a system, that allows delivery of these components into a targeted cell and nucleus. By allowing the delivery of the genome-modifying enzymes into target cells only, the methods of the invention can be used not only in cell cultures, but can be used when the cell population in a target is mixed, such as, e.g., mammalian, such as murine or human tissues. The methods of the invention can be used to targeted modification of, e.g., specific neural cells that express a particular marker that can be targeted. The bacterial toxin fused to the genome modifying enzyme can be targeted to such cell. Moreover, if the system utilizing two different enzymes is used, and again targeted to the specific cell, the accuracy of the genome editing in only the desired cells can be increased.

The system can therefore be used for in vivo genome editing.

Methods for engineering fusion proteins, such as those used in the methods of the present invention are well known to one of ordinary skill in the art. For example, one can use native chemical ligation or sortase-mediated protein ligation which has been described, e.g., in international patent application publication No. PCT/US/2013/027307 and Thomas Proft, Sortase-mediated protein ligation: an emerging biotechnology tool for protein modification and immobilization. Biotechnol Letters 32:1-10, 2010. Methods of engineering short peptides to incorporate pore forming protein toxin recognition sequences, such as anthrax toxin lethal factor N-terminal domain (LFn), described, e.g., in, Boll et al. Eur J Cell Biol. 2004 July; 83(6):281-8, incorporated herein by reference in its entirety, have also been described, e.g., in U.S. Patent Application Publication No. 20130336974, incorporated herein by reference. The sequence of “LF” or “anthrax toxin lethal factor” is known in the art, e.g., NCBI GeneID Nos: 3361711 or 33617126; e.g. NCBI Ref Seq: NP_052803 or NP_052818. In some embodiments, the protein tag capable of binding to a pore forming protein can be LF. In some embodiments, the protein tag can be a portion of LF, e.g., LFn (e.g. at least residues 1-295 of LF).

Recombinant production of enzymes can also be used to incorporate the tag to the compositions of the invention. Fusion gene is first made by combining a nucleic acid encoding the enzyme with a nucleic acid encoding a suitable tag in a suitable expression vector and expressing such fusion construct to make the engineered compositions of the invention.

In the methods and kits of the invention, one can use or include composition comprising a pore forming protein conjugated to a cellular target signal. The composition may be in the form of a peptide or a nucleic acid expressing the peptide. For instance the composition may be a nucleic acid expression vector including the elements for expressing the pore forming protein conjugated to a cellular target signal. A variety of suitable vectors are available for expressing genetic material in cells. The selection of an appropriate vector to deliver a therapeutic agent for a particular condition and the optimization of the conditions for insertion of the selected expression vector into the cell, are within the scope of the skilled artisan.

As used herein, a “vector” may be any of a number of nucleic acid molecules into which a desired sequence may be inserted by restriction and ligation for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript.

In some aspects of the invention, we provide a composition comprising a nucleic acid encoding the engineered fusion protein.

The term “protein tag capable of binding to a pore forming protein” as used herein includes any protein tag that can be used to recognize a pore forming protein. For example, N-terminal fragment of the anthrax toxin lethal factor LFn can be used.

The enzyme may further comprise an additional localization signal or targeting peptide to allow delivery to any cellular compartment. A targeting peptide is typically a short (3-70 amino acids long) peptide chain that directs the transport of a protein to a specific region in the cell, including the nucleus, mitochondria, endoplasmic reticulum (ER), chloroplast, apoplast, peroxisome and plasma membrane. Some target peptides are cleaved from the protein by signal peptidases after the proteins are transported.

For example, a nuclear localization signal (NLS) is a target peptide that directs proteins to the nucleus and is often a unit consisting of five basic, positively-charged amino acids. The NLS normally is located anywhere on the peptide chain.

As another example, the mitochondrial targeting signal is a 10-70 amino acid long peptide that directs a newly synthesized proteins to the mitochondria. It is found at the N-terminus and consists of an alternating pattern of hydrophobic and positively charged amino acids to form what is called an amphipathic helix. Mitochondrial targeting signals can contain additional signals that subsequently target the protein to different regions of the mitochondria, such as the mitochondrial matrix. Like signal peptides, mitochondrial targeting signals are cleaved once targeting is complete.

Examples of intracellular localization signals are shown below

| | |
|---|---|
| Transport to | H2N-Met-Leu-Ser-Leu-Arg-Gln- |
| the mitochondrial | Ser-Ile-Arg-Phe-Phe-Lys-Pro- |
| matrix | Ala-Thr-Arg-Thr-Leu-Cys-Ser- |
| | Ser-Arg-Tyr-Leu-Leu- |
| | (SEQ ID NO: 2) |
| Transport to the | -Pro-Pro-Lys-Lys-Lys-Arg-Lys- |
| nucleus (NLS) | Val- (SEQ ID NO: 3) |

For example, the enzyme can be a zinc-finger nuclease. Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to precisely alter the genomes of higher organisms.

A transcription activator-like effector nuclease, or TALENs, are artificial restriction enzymes generated by fusing a TAL effector DNA binding domain to a DNA cleavage domain. Typically, each TALEN monomer consists of the TAL effector DNA binding domain with a FokI catalytic domain fused to its C terminus. The monomers are designed to bind to candidate target sites (red on the image above) oriented from 5′ to 3′ on opposite strands of DNA. The spacer region between the sites must be sufficiently large enough for the two FokI domains to dimerize and cut the DNA, but not so long that they do not come into contact.

Monomeric site specific nucleases can be used in the methods of the invention. Precise genome editing in complex genomes is enabled by engineered nucleases that can be programmed to cleave in a site-specific manner.

For example, a monomeric nuclease domain derived from GIY-YIG homing endonucleases for genome-editing applications. Fusion of the GIY-YIG nuclease domain to three-member zinc-finger DNA binding domains generated chimeric GIY-zinc finger endonucleases (GIY-ZFEs). Significantly, the I-TevI-derived fusions (Tev-ZFEs) function in vitro as monomers to introduce a double-strand break, and discriminate in vitro and in bacterial and yeast assays against substrates lacking a preferred 5′-CNNNG-3′ cleavage motif. The Tev-ZFEs function to induce recombination in a yeast-based assay with activity on par with a homodimeric Zif268 zinc-finger nuclease. We also fused the I-TevI nuclease domain to a catalytically inactive LADGLIDADG homing endonuclease (LHE) scaffold (“LADGLIDADG” disclosed as SEQ ID NO: 4). The monomeric Tev-LHEs are active in vivo and similarly discriminate against substrates lacking the 5′-CNNNG-3′ motif. The monomeric Tev-ZFEs and Tev-LHEs are distinct from the Fold-derived zinc-finger nuclease and TAL effector nuclease platforms as the GIY-YIG domain alleviates the requirement to design two nuclease fusions to target a given sequence, highlighting the diversity of nuclease domains with distinctive biochemical properties suitable for genome-editing applications. (Kleinstiver et al., Proc Natl Acad Sci USA. 2012 May 22; 109(21):8061-6. Epub 2012 May 7).

Another example is a fusion of the small, sequence-tolerant monomeric nuclease domain from the homing endonuclease I-TevI to transcription-like activator effectors (TALEs) to create monomeric Tev-TALE nucleases (Tev-mTALENs). (Kleinstiver et al., G3, Genes, Genomes, Genetics, Early Online Apr. 16, 2014, doi: 10.1534/g3.114.011445).

Naturally occurring examples of sets of pore forming proteins and pore specific delivery protein exist. For example many bacterial toxins include a pore forming protein and a pore specific delivery protein, either together within a single protein or in separate proteins that function together. Diphtheria toxin, for example, is a single protein containing both a pore forming protein and a pore specific delivery protein. In contrast, anthrax toxin is composed of multiple peptides which make up the pore forming protein (referred to as protective antigen or PA in anthrax toxin) and a pore specific delivery protein (edema factor (EF) or lethal factor (LF) in anthrax toxin). Naturally occurring toxins that include these peptides useful in the methods of the invention include but are not limited to anthrax toxin, diphtheria toxin, pertussis toxin, cholera toxin, botulinum neurotoxin, shiga toxin, shiga like toxin, pseudomonas exotoxin, tetanus toxin, and exotoxin A. The pore forming protein may be a naturally occurring toxin pore forming protein or may be a modified pore forming protein, that includes one or more non-naturally occurring entities.

Examples of the naturally occurring pore forming proteins include any known pore forming toxin, such as Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.

The pore specific binding peptide is a peptide that interacts with a pore in a manner that enables transport of the peptide and any related attached cargo through the pore. While the pore specific binding peptide interacts with the pore sequence a variety of peptide sequences that vary from the naturally occurring sequence can be used. Thus, the pore specific binding peptide may be a fragment of a naturally occurring toxin, a variant thereof or a synthetic peptide sequence. An exemplary pore specific binding peptide has an amino acid sequence comprising:

X1-′Y1-X2-′Y2-X3-′Y3-X4-′Y4
or
Yi-Xi-Y2-X2-Y3-X3-Y_{4-X4}

wherein X is a negatively charged amino acid and Y is a positively charged L amino acid, and X′ and Y′ are non-natural entities, including D-amino acids or other chemical entities, with the ionization properties corresponding to X and Y. In some embodiments, X1, X2, X3, and X4 are selected from E and D or D-amino acid isoforms of E and D. In other embodiments Yi, Y2, Y3, and Y4 are selected from K, R, and H, or D-amino acid isoforms of K, R, and H. In some embodiments the pore specific binding peptide is a peptide of 8-50 amino acids in length. Alternatively, the peptide may be 10-40, 15-30, or 20-25 amino acids in length.

In some aspects, the pore forming protein further comprises a cell targeting peptide. Such peptide can be added to the pore-forming protein using protein ligation, such as sortase-mediated ligation or recombinant fusion protein production.

A linker may be used to connect the pore specific binding peptide and the complex biomolecule. The linker may optionally be susceptible to cleavage in the cytosolic compartment. Linker molecules (“linkers”) may be peptides, which consist of one to multiple amino acids, or non-peptide molecules. Examples of peptide linker molecules useful in the invention include glycine-rich peptide linkers (see, e.g., US 5,908,626), wherein more than half of the amino acid residues are glycine. Preferably, such glycine-rich peptide linkers consist of about 20 or fewer amino acids.

Linker molecules may also include non-peptide or partial peptide molecules. For instance the peptide may be linked to other molecules using well known cross-linking molecules such as glutaraldehyde or EDC (Pierce, Rockford, Ill.). Bifunctional cross-linking molecules are linker molecules that possess two distinct reactive sites. For example, one of the reactive sites of a bifunctional linker molecule may be reacted with a functional group on a peptide to form a covalent linkage and the other reactive site may be reacted with a functional group on another molecule to form a covalent linkage.

General methods for cross-linking molecules have been reviewed (see, e.g., Means and Feeney, Bioconjugate Chem., 1: 2-12 (1990)).

Homobifunctional cross-linker molecules have two reactive sites which are chemically the same. Examples of homobifunctional cross-linker molecules include, without limitation, glutaraldehyde; N,N′-bis(3-maleimido-propionyl-2-hydroxy-1,3-propanediol (a sulfhydryl-specific homobifunctional cross-linker); certain N-succinimide esters (e.g., discuccinimyidyl suberate, dithiobis(succinimidyl propionate), and soluble bis-sulfonic acid and salt thereof (see, e.g., Pierce Chemicals, Rockford, Ill.; Sigma-Aldrich Corp., St. Louis, Mo.).

Preferably, a bifunctional cross-linker molecule is a heterobifunctional linker molecule, meaning that the linker has at least two different reactive sites, each of which can be separately linked to a peptide or other molecule. Use of such heterobifunctional linkers permits chemically separate and stepwise addition (vectorial conjunction) of each of the reactive sites to a selected peptide sequence. Heterobifunctional linker molecules useful in the invention include, without limitation, m-maleimidobenzoyl-N-hydroxysuccinimide ester (see, Green et al., Cell, 28: 477-487 (1982); Palker et al., Proc. Natl. Acad. Sci (USA), 84: 2479-2483 (1987)); m-maleimido-benzoylsulfosuccinimide ester; maleimidobutyric acid N-hydroxysuccinimide ester; and N-succinimidyl 3-(2-pyridyl-dithio)propionate (see, e.g., Carlos et al., Biochem. J., 173: 723-737 (1978); Sigma-Aldrich Corp., St. Louis, Mo.).

When it is desirable to deliver the reagent to a specific cell, the reagent may be targeted to a specific type of cell or tissue. Typically, the pore forming protein is bound to a cellular target signal. A cellular target signal as used herein is a molecule which specifically recognizes and binds to a cell surface molecule associated with a specific type of cell or tissue. For example the cellular target signal may recognize and bind to a cell surface receptor and as such is referred to as a cell surface receptor binding peptide. Cell surface binding peptides include but are not limited to peptides that bind Her2, tumor necrosis factor receptor (TNFR), cytotoxic T lymphocyte antigen 4 (CTLA4), programmed cell death protein 1 (PD1), B- and T lymphocyte attenuator (BTLA), lymphocyte activation gene 3 (LAG3), CD160, PD1 homolog (PD1H), CD28, inducible co-stimulator (ICOS), CD137 (also known as 4-1BB), CD27, OX40, glucocorticoid-induced TNFR-related protein (GITR), CD40 ligand (CD40L), B cell activation factor receptor (BAFFR), transmembrane activator, CAML interactor (TACI), B cell maturation antigen (BCMA), B7 ligand members, APRIL, a proliferation-inducing ligand; B7H1, B7 homolog 1; GITRL, GITR ligand; HVEM, herpesvirus entry mediator; IT AM, immunoreceptor tyrosine-based activation motif; ITIM, immunoreceptor tyrosine-based inhibitory motif; ITSM, immunoreceptor tyrosine-based switch motif; MHC, major histocompatibility complex; OX40L, OX40 ligand; PI3K, phosphoinositide 3-kinase; TCR, T cell receptor; TRAF, or TNFR-associated factor binding peptide.

A variety of specific cell receptors can be targeted using the compositions and methods of the present embodiments, as long as the receptor is one of those that internalize their ligands and traffic them to an acidic intracellular compartment, which facilitates proper folding of the translocated components. Receptors that can be targeted by the engineered binary toxins according to the present invention include, for example, HER1, HER2, HER3 and HER4 EGF receptors; vascular endothelial growth factor receptors VEGFR-1, VEGFR-2 and VEGFR-3; insulin-like growth factor 1 receptors; fibroblast growth factor receptors; thrombospondin 1 receptors; estrogen receptors; urokinase receptors; progesterone receptors; testosterone receptors; carcinoembryonic antigens; prostate-specific antigens; farnesoid X receptors; transforming growth factor receptors; transferrin receptors; hepatocyte growth factor receptors; or vasoactive intestinal polypeptide receptors 1 and 2. Neuronal cells can be targeted by targeting neuronal receptors such as adrenergic receptors, dopaminergic receptors, GABAerigic receptors, glutaminergic receptors, histaminergic receptors, cholinergic receptors, opioid receptors, serotonergic receptors or glycinergic receptors. One can also target receptors on specific neurons, such as the MRG gene family (also known as SNSR) which belongs to the G-protein-coupled receptor (GPCR) superfamily, and is expressed specifically in nociceptive neurons, and is implicated in the modulation of nociception.

The invention also relates to compositions that are useful according to the methods of the invention. An exemplary composition of the invention is a fusion molecule of a pore specific delivery protein linked to the complex biomolecule, wherein the complex biomolecule is delivered by itself, and may further comprise an intracellular localization signal peptide, a labeled compound, a halogenated compound, a morpholino, a therapeutic RNA, a protein mimic, antibody mimic, a mirror image biomolecule or a monobody, or an engineered protein scaffold.

The biomolecule for instance, may be linked to a PEG molecule. Such a molecule is referred to as a PEGylated protein.

In some aspects, the invention is a method for delivering an enzyme, such as a genome modifying enzyme, to the cytosol, nucleus or another cellular compartment of a targeted living cell, by contacting the targeted living cell with a pore forming protein, wherein the pore forming protein has a cellular target signal, wherein the cellular target signal targets the pore forming protein to the targeted living cell, and a fusion molecule comprising a pore specific delivery protein pore specific delivery protein linked to the enzyme, wherein the enzyme is delivered to the cytosol or nucleus or other cellular compartment of the targeted living cell.

The cell-targeting peptide can be selected, for example, from a cell receptor ligand, an antibody, or an affibody.

In some aspects of all the embodiments of the invention, the cell receptor-binding ligand specific for a target cell comprises an antibody and/or an antigen-binding portion or fragment of an antibody.

An example antibody to target HER2 is trastuzumab, a recombinant monoclonal antibody used in therapeutics (HERCEPTIN). Another monoclonal antibody that targets HER2 positive cells and prevents dimerization of HER2 and HER3 is Pertuzumab (also called 2C4, trade name PERJETA). Either one of these antibodies can be used to fuse with the receptor ablated PA86 subunit, such as the mPA.

The term “antibody” refers to an immunoglobulin (e.g., IgG, IgM, IgA, IgE, IgD, etc.).

The basic functional unit of each antibody is an immunoglobulin (Ig) monomer (containing only one immunoglobulin (“Ig”) unit). Included within this definition are monoclonal antibodies, chimeric antibodies, recombinant antibodies, and humanized antibodies.

In one embodiment, the invention's antibodies are monoclonal antibodies produced by hybridoma cells.

In particular, the invention contemplates antibody fragments that contain the idiotype (“antigen-binding fragment”) of the antibody molecule. For example, such fragments include, but are not limited to, the Fab region, F(ab′)2 fragment, pFc′ fragment, and Fab′ fragments.

The “Fab region” and “fragment, antigen binding region,” interchangeably refer to portion of the antibody arms of the immunoglobulm “Y” that function in binding antigen. The Fab region is composed of one constant and one variable domain from each heavy and light chain of the antibody. Methods are known in the art for the construction of Fab expression libraries (Huse et al., Science, 246: 1275-1281 (1989)) to allow rapid and easy identification of monoclonal Fab fragments with the desired specificity. In another embodiment, Fc and Fab fragments can be generated by using the enzyme papain to cleave an immunoglobulin monomer into two Fab fragments and an Fc fragment. The enzyme pepsin cleaves below the hinge region, so a “F(ab′)2 fragment” and a “pFc′ fragment” is formed. The F(ab′)2 fragment can be split into two “Fab′ fragments” by mild reduction.

The invention also contemplates a “single-chain antibody” fragment, i.e., an amino acid sequence having at least one of the variable or complementarity determining regions (CDRs) of the whole antibody, and lacking some or all of the constant domains of the antibody. These constant domains are not necessary for antigen binding, but constitute a major portion of the structure of whole antibodies. Single-chain antibody fragments are smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. Techniques for the production of single-chain antibodies are known (U.S. Pat. No. 4,946,778). The variable regions of the heavy and light chains can be fused together to form a “single-chain variable fragment” (“scFv fragment”), which is only half the size of the Fab fragment, yet retains the original specificity of the parent immunoglobulm.

The “Fc” and “Fragment, crystallizable” region interchangeably refer to portion of the base of the immunoglobulin “Y” that function in role in modulating immune cell activity. The Fc region is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils and eosinophils. In an experimental setting, Fc and Fab fragments can be generated in the laboratory by cleaving an immunoglobulin monomer with the enzyme papain into two Fab fragments and an Fc fragment.

In some aspects of all the embodiment of the invention, the non-toxin-associated receptor-binding ligand specific for a target cell comprises an affibody.

“Affinity body,” “Affibody®,” and “affibody” molecules are antibody mimetic proteins that, like antibodies, can specifically bind target antigens (Nord, K., et al. (1997) Nature Biotechnol. 15: 772-777). Affibody molecules can be designed and used like aptamers. In one embodiment, Affibody molecules comprise a backbone derived from an IgG-binding domain of Staphylococcal Protein A (Protein A produced by S. aureus). The backbone can be derived from an IgG binding domain comprising the three alpha helices of the IgG-binding domain of Staphylococcal Protein A termed the B domain. The amino acid sequence of the B domain is described in Uhlen et al, J. Biol. Chem. 259: 1695-1702 (1984). Alternatively, the backbone can be derived from the three alpha helices of the synthetic IgG-binding domain known in the art as the Z domain, which is described in Nilsson et al., Protein Eng. 1: 107-113 (1987). The backbone of an affibody comprises the amino acid sequences of the IgG binding domain with amino acid substitutions at one or more amino acid positions. The affibody, for example, comprises the 58 amino acid sequence of the Z domain (VDNKFDKEXXXAXXEIXXLPNLNXXQXXAFIXSLXDDPSQSADLLAEAKKLDDAQAPK, SEQ ID NO: 5), wherein X at each of positions 9, 10, 11, 13, 14, 17, 18, 24, 25, 27, 28, 32, and 35 is any amino acid (Capala et al, U.S. Pat. Appl. No. US20100254899).

The affibody molecule constitutes a highly suitable carrier for directing molecules of interest (e.g., toxins, radioisotopes, therapeutic peptides) to, e.g., tumor cells due to specific target binding and lack of irrelevant interactions, such as the Fc receptor binding displayed by some antibodies.

Common advantages of AFFIBODY® molecules over antibodies are better solubility, tissue penetration, stability towards heat and enzymes, and comparatively low production costs.

Affibodies are exemplified by, but not limited to, Anti-ErbB2 AFFIBODY® (also referred to as anti-HER2 AFFIBODY®), Anti-EGFR AFFIBODY®, Anti-TNF alpha AFFIBODY®, Anti-fibrinogen AFFIBODY®, Anti-transferrin AFFIBODY®, Anti-HSA AFFIBODY®, Anti-Insulin AFFIBODY®, Anti-IgG AFFIBODY®, Anti-IgM AFFIBODY®, Anti-IgA AFFIBODY®, and Anti-IgE AFFIBODY® (e.g., from Abceam, Cambridge, Mass.).

Affibodies with an affinity of down to sub-nanomolar have been obtained from naive library selections, and affibodies with picomolar affinity have been obtained following affinity maturation (Orlova et al. (2006). “Tumor imaging using a picomolar affinity HER2 binding affibody molecule”. Cancer Res. 66 (8): 4339-48. PMID 16618759). Affibodies conjugated to weak electrophiles bind their targets covalently (Holm et al., Electrophilic affibodies forming covalent bonds to protein targets, J Biol Chem. 2009 Nov. 20; 284(47):32906-13. PMID 19759009).

Affibody molecules can be synthesized chemically or in bacteria or purchased from a commercial source (e.g., Affibody AB, Bromma, Sweden; Abeam, Cambridge, Mass.).

Affibody molecules can also be obtained by constructing a library of affibodies as described in U.S. Pat. No. 5,831,012, which is incorporated herein by reference. The affibody library can then be screened for affibodies which bind to target antigens of interest (e.g., HER-2, EGFR) by methods known in the art.

Affibody molecules are based on a three-helix bundle domain, which can be expressed in soluble and proteolytically stable forms in various host cells on its own or via fusion with other protein partners (Stahl et al. (1997). “The use of gene fusions to protein A and protein G in immunology and biotechnology”. Pathol. Biol. (Paris) 45: 66-76. PMID 9097850.”

Affibodies tolerate modification and are independently folding when incorporated into fusion proteins. Head-to-tail fusions of Affibody molecules of the same specificity have proven to give avidity effects in target binding, and head-to-tail fusion of Affibody molecules of different specificities makes it possible to get bi-specific or multi-specific affinity proteins. Fusions with other proteins can also be created (Ronnmark et al. (2002) “Construction and characterization of affibody-Fc chimeras produced in Escherichia coli,” J. Immunol Methods 261: 199-211. PMID 11861078; Ronnmark et al. (2003) “Affibody-beta-galactosidase immunoconjugates produced as soluble fusion proteins in the Escherichia coli cytosol,” J. Immunol Methods 281: 149-160. PMID 14580889). A site for site-specific conjugation is facilitated by introduction of a single cysteine at a desired position.

A number of different Affibody molecules have been produced by chemical synthesis. Since they do not contain cysteines or disulfide bridges, they fold spontaneously and reversibly into the correct three-dimensional structures when the protection groups are removed after synthesis (Nord et al. (2001) “Recombinant human factor V111-specific affinity ligands selected from phage-displayed combinatorial libraries of protein A,” Eur. J. Biochem. 268: 1-10. PMID 11488921; Engfeldt et al. (2005) “Chemical synthesis of triple-labeled three-helix bundle binding proteins for specific fluorescent detection of unlabeled protein,” Chem. BioChem. 6: 1043-1050. PMID 15880677).

The invention provides a method for site-specific modification of the genome of a mammalian cell comprising the steps of contacting the mammalian cell with a pore forming protein and the engineered enzyme, such as genome modifying enzyme.

Accordingly, the invention provides methods for genetic manipulation of cells carrying specific receptors by targeting enzymes to these cells. In these methods, the cell is contacted with both a pore-forming unit of the toxin which is targeted to a specific receptor and at least one enzyme capable of genetic modification. Additionally, the cell can be contacted with one or more nucleic acids encoding proteins, RNAs or other genetic components one may wish to add to the specific site allowed by the genome modifying enzyme.

The native receptor-binding ligand of the toxin pore forming unit is typically ablated or replaced and the toxin pore forming unit is fused with a ligand, antibody or affibody that can bind specifically to a receptor on a target cell, e.g., a cancer cell or an immune cell or neuron to allow targeting of the pores to particular cell populations. Thus, the pore forming unit retains determinants needed for the cytoplasmic delivery of the pore forming unit binding units, but specific cell targeting can be selected. The native pore binding component contains the catalytic activity, and allows translocation to the target cell cytosol via the pore forming unit or component.

The cell can be any cell, including natural and artificial cells. Thus, the invention, in some aspects is a method for delivering a reagent to the interior of a living cell. The cell may be any type of living cell. For example living cells include eukaryotic cells and prokaryotic cells. For example the cell can be a eukaryotic cell, such as a mammalian cell, such as human, murine, simian, canine, feline, bovine, or avian cell. Examples of living cells include but are not limited to cells derived from humans, primates, dogs, cats, horses, cows, pigs, turkeys, goats, fish, monkeys, chickens, rats, mice, sheep, plants, bacteria, algae, and yeast. The cells may be normal cells, pre-malignant cells, malignant cells, or genetically engineered cells. Moreover, the cell can be either isolated cell in vitro, or part of a tissue or organ, such as blood, heart, brain, central nervous system, kidney, liver, lungs, bone, muscle, eye, ear, or skin, in vivo. The cell can also be part of a tissue, ex vivo, such as blood. In some aspects, the cell is a neuronal system cell. In some aspects the cell is brain cell.

In some aspects, the cell is contacted with at least two pairs of the engineered proteins, such as enzymes, such as genome modifying enzymes, wherein each of the at least two pairs of the engineered site-specific nucleases target a different receptor and are active only when transported via the different receptors into the same mammalian cell, thus minimizing both off-target activity and activity in non-target cells.

In some aspects, the off-target activity comprises cutting DNA at a single TALEN binding site.

In some aspects, the at least two pairs of the engineered site specific nucleases comprise a TALEN and a CRISPR nuclease targeted to adjacent sequences in which FokI nuclease dimerization is required for cutting.

CRISPRs (clustered regularly interspaced short palindromic repeats) are DNA loci containing short repetitions of base sequences. Each repetition is followed by short segments of “spacer DNA” from previous exposures to a virus. CRISPRs are often associated with cas genes that code for proteins related to CRISPRs. The CRISPR/Cas system is a prokaryotic immune system that confers resistance to foreign genetic elements such as plasmids and phages (Barrangou, R. et al., Science 315 (5819): 1709-1712; Marraffini, L. A.; Sontheimer, E. J. (2008), Science 322 (5909): 1843-1845) and provides a form of acquired immunity CRISPR spacers recognize and silence these exogenous genetic elements like RNAi in eukaryotic organisms. (Marraffini, L. A.; Sontheimer, E. J. (2010) Nature Reviews Genetics 11 (3): 181-190). Since 2012, the CRISPR/Cas system has been used for gene editing (silencing, enhancing or changing specific genes) that even works in eukaryotes like mice and primates. CRISPR nucleases also include clustered regulatory interspaced short palindromic repeat (CRISPR)/Cas-based RNA-guided DNA endonucleases.

Another example is FokI nuclease. The type IIs restriction endonuclease FokI, isolated from Flavobacterium okeanokoites, recognizes an asymmetric nucleotide sequence and cleaves both DNA strands outside of the recognition site: 5′-GGATG(N)9/13 (1). The fokIRM genes have been cloned and sequenced (Looney M, et al., (1989) Gene 80:193-208; Kita K, et al., (1989) J Biol Chem 264:5751-5756, pmid:2784436). The endonuclease consists of 587 aa with a molecular mass of 65.4 kDa (Looney M, et al., (1989) Gene 80:193-208).

Accordingly, in some aspects, the method comprises contacting the cell with a nicking version of the FokI nuclease in TALE or Cas9 fusions targeted to adjacent sequences, wherein the targeted nucleases bind and nick DNA separately, and a double strand break forms if the second cut was made before the first was repaired.

In some aspects, the method comprises protein complementation, wherein at least one of the nucleases is divided into two separate and inactive domains which are fused to TALE or Cas9 proteins, wherein DNA double strand breaks occur only when the fusions bind to their target sequences in the correct orientation.

In some aspects, the method further comprises a step of contacting the cell with a replacement nucleic acid.

In some aspects of all the embodiments and aspects of the invention, the method is performed in vivo, in vitro or ex vivo.

Engineered Zn-finger nucleases (ZFNs) were the first example of site-specific nucleases to be used for targeted cleavage of genomic sequences. ZFNs are composite proteins made up of the sequence-specific DNA binding domain derived from a Zn-finger transcription factor fused to a non-specific DNA endonuclease domain, for example, from the FokI endonuclease, and a nuclear localization signal. ZFNs have largely been supplanted by the more recently developed transcription activator-like effector nucleases (TALENs) as tools for genome modification. TALEN proteins are constructed in the same manner as ZFNs with a FOK I endonuclease domain and nuclear localization signal fused with the sequence-specific DNA binding domain—in this case borrowed from the transcription-activation effector proteins injected by Xanthamonas sp. into host (plant) cells. TALENs can be programmed to bind to and cut specific DNA sequences by simply changing the copy number and composition of a repeated 34 amino acid domain. Transfection experiments have shown that TALENs, when used in pairs that have been designed to recognize adjacent target sequences of sufficient length (18-20 bp), can create double-strand DNA breaks at a single site in the human genome.

DNA double-strand breaks, whether they are created by TALENs or ZFNs or by other means, are repaired in mammalian and other eukaryotic cells by either non-homologous end-joining (NHEJ), or homologous recombination pathways. Looked at from an engineering perspective, each of these repair modes enables important applications for the manipulation of genomic sequences. Repair of double strand breaks by NHEJ pathways generally results in the addition or loss of DNA surrounding the break, and therefore can result in gene knockout if the break repair interrupts coding sequence of a gene. Repair of double strand breaks by homologous recombination, which is generally an error free process, enables gene replacement provided that DNA breaks are introduced at both ends of the chromosomal sequence to be replaced, and the transfected DNA has ends with sufficient homology to the ends of the chromosomal strand breaks.

The invention provides pharmaceutical compositions and kits comprising the engineered enzyme alone or together with a pore-forming unit of a toxin it is designed to pair with.

The kits may comprise one or more containers comprising one or more engineered enzymes and the corresponding engineered pore-forming toxin units, e.g., units that have been engineered to target a particular extracellular receptor.

The pharmaceutical composition further comprises a pharmaceutically acceptable carrier.

The compositions may be delivered to a subject, a tissue, or a cell in a carrier or a pharmaceutically acceptable carrier. A subject may be a human subject or a non-human subject. As used herein, the terms “pharmaceutically acceptable” refers to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation.

Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

“Pharmaceutically acceptable carrier” as used herein includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The compounds may be sterile or non-sterile.

The compounds described herein may comprise different types of carriers depending on whether it is to be administered in solid, liquid or aerosol form, and whether it need to be sterile for such routes of administration as injection. The present invention can be administered intravenously, intratracheally directly into the brain or a brain region, e.g., under stereotactic guidance, intradermally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue or organ. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients. In general, a pharmaceutical composition comprises the compound of the invention and a pharmaceutically acceptable carrier. Pharmaceutically acceptable carriers for nucleic acids, small molecules, peptides, monoclonal antibodies, and antibody fragments are well-known to those of ordinary skill in the art. As used herein, a pharmaceutically acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants, including those designed for slow or controlled release.

Compositions suitable for oral administration may be presented as discrete units, such as capsules, tablets, lozenges, each containing a predetermined amount of the active agent. Other compositions include suspensions in aqueous liquids or nonaqueous liquids, such as a syrup, an elixir or an emulsion.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

The invention also includes kits made up of the various reagents described herein assembled to accomplish the methods of the invention. A kit for instance may include a biomolecule fused to the pore-specific binding peptide, one or more pore forming proteins, optionally linked to a target binding peptide. The kit may further comprise assay diluents, standards, controls and/or detectable labels. The assay diluents, standards and/or controls may be optimized for a particular sample matrix. Reagents include, for instance, antibodies, nucleic acids, labeled secondary agents, or in the alternative, if the primary reagent is labeled, enzymatic or agent binding reagents which are capable of reacting with the labeled reagent. One skilled in the art will readily recognize that reagents of the present invention can be readily incorporated into one of the established kit formats which are well known in the art.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment or characterization of a cancer.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The phrase “consisting essentially of” is intended to encompass the components necessary for the function of the method or composition and any non-functional added components, such as buffers, containers, and other non-essential components. The phrase “consisting of is intended to include only the components as listed.

As used herein, “nuclease” refers to an enzyme capable of cleaving the phosphodiester bonds between the nucleotide subunits of nucleic acids. Nucleases can be site-specific, i.e. site-specific nucleacses cleave DNA bonds only after specifically binding to a particular sequence. Therefore, nucleases specific for a given target can be readily selected by one of skill in the art. Nucleases often cleave both strands of dsDNA molecule within several bases of each other, resulting in a double-stranded break (DSB). Exemplary nucleases include, but are not limited to Cas9; meganucleases; TALENS; zinc finger nucleases; FokI cleavage domain; RNA-guided engineered nucleases; Cas9-derived nucleases: homing endonucleases(e.g. I-AniI, I-CreI, and I-SceI) and the like. Further discussion of the various types of nucleases and how their site-specificity can be engineered can be found, e.g. in Silva et al, Curr Gene Ther 2011 11:11-27; Gaj et al. Trends in Biotechnology 2013 31:397-405; Humbert et al. Critical Reviews in Biochemistry and Molecular Biology 2012 47:264-281; and Kim and Kim Nature 2014 doi:10.1038/nrg3686; each of which is incorporated by reference herein in its entirety.

As used herein, “recombinase” refers to a site-specific enzyme that recognizes short DNA sequence(s), which sequence(s) are typically between about 30 base pairs (bp) and 40 bp, and that mediates the recombination between these recombinase recognition sequences, which results in the excision, integration, inversion, or exchange of DNA fragments between the recombinase recognition sequences. By way of non-limiting example, recombinases can include serine recombinases (e.g., resolvases and invertases) and tyrosine recombinases (e.g., integrases). Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, φC31 (phiC31), TP901, TGI, φBTI, R4, cpRV1, cpFC1, MRU, A118, U153 and gp29. Examples of bidirectional tyrosine recombinases include, without limitation, Cre, FLP, and R; and unidirectional tyrosine recombinases include, without limitation, Lambda, HKlO1, HK022 and pSAM2.

As used herein, “cell-targeting” refers to an entity or moiety (e.g. a peptide) that targets a particular cell type. Cell-targeting constructs can target a cell, by interacting with, or binding to, cell-surface receptors or other molecules on the cell surface. Cell targeting constructs also can target cells by interacting with proteins secreted by the cell. For example, a cell targeting peptide can binds to a cell surface receptor of a cell or the cell-targeting peptide can be cleaved by a protease residing on the surface of a cell, or a protease secreted by a cell (and, thus, is concentrated in the locality of the cell).

As used herein, “pore-forming protein” refers to a transmembrane protein that naturally comprises at least a pore (i.e. channel). In some embodiments, the pore-forming protein is capable of forming a pore spanning the entire length of a membrane bilayer.

As used herein, “nuclear localization signal” refers to a peptide (or a nucleic acid sequence encoding such a peptide) that when present as part of a larger polypeptide comprising a cargo polypeptide, will localize the cargo polypeptide to a specific subcellular location, i.e., the nucleus. As used herein, a cargo polypeptide is “localized” to a particular subcellular location by a localization signal, when transcribed with that operably linked signal, its concentration at that subcellular location is at least 10% greater than without the operably linked signal or tag, e.g. at least 10%, at least 20%, at least 30%, at least 50%, at least 75%, at least 100%, at least 200%, or at least 500% or greater than without the operably linked signal or tag.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

Some embodiments of the technology described herein can be defined according to any of the following numbered paragraphs:
1. An engineered site-specific nuclease protein comprising a site-specific nuclease protein and a protein tag capable of binding to a pore forming protein.
2. The engineered site-specific nuclease protein of paragraph 1, further comprising a nuclear localization signal.
3. The engineered site-specific nuclease protein of paragraphs 1 or 2, wherein the site-specific nuclease is selected from a zinc-finger nuclease, a transcription activator-like effector nuclease, and a monomeric site-specific nuclease.
4. The engineered site-specific nuclease protein of paragraph 3, wherein the monomeric site-specific nuclease is an engineered GIY-YIG family protein.
5. The engineered site-specific nuclease protein of any one of paragraphs 1-4, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
6. A method for delivering an engineered site-specific nuclease into a mammalian cell comprising contacting the mammalian cell with a pore forming protein and the engineered site-specific nuclease of any one of the paragraphs 1-5.
7. The method of paragraph 6, wherein the pore forming protein further comprises a cell-targeting peptide.
8. The method of paragraph 7, wherein the cell-targeting peptide is selected from a cell receptor ligand, an antibody, or an affibody.
9. A method for site-specific modification of the genome of a mammalian cell comprising the steps of contacting the mammalian cell with a pore forming protein and the engineered site-specific nuclease of any one of the paragraphs 1-5.
10. The method of paragraph 9, wherein the core forming protein further comprises a cell targeting peptide.
11. The method of paragraph 10, wherein the cell-targeting peptide is selected from a cell receptor ligand, an antibody, or an affibody.
12. The method of paragraph 8, wherein the mammalian cell is contacted with at least two pairs of the engineered site-specific nucleases of any one of paragraphs 1-5, wherein each of the at least two pairs of the engineered site-specific nucleases target a different receptor and are active only when transported via the different receptors into the same mammalian cell, thus minimizing both off-target activity and activity in non-target cells.
13. The method of paragraph 12, wherein the off-target activity comprises cutting DNA at a single TALEN binding site.
14. The method of paragraph 12, wherein the at least two pairs of the engineered site specific nucleases comprise a TALEN and a CRISPR nuclease targeted to adjacent sequences in which FokI nuclease dimerization is required for cutting.
15. The method of paragraph 12, comprising use of a nicking version of the FokI nuclease in TALE or Cas9 fusions targeted to adjacent sequences, wherein the targeted nucleases bind and nick DNA separately, and a double strand break forms if the second cut was made before the first was repaired.
16. The method of paragraph 14, comprising protein complementation, wherein at least one of the nucleases is divided into two separate and inactive domains which are fused to TALE or Cas9 proteins, wherein DNA double strand breaks occur only when the fusions bind to their target sequences in the correct orientation.
17. The method of any one of paragraphs 9-16 and 130-134, further comprising a step of contacting the cell with a replacement nucleic acid.
18. The method of any one of the paragraphs 6-17 and 130-134, wherein the mammalian cell is a neuronal system cell.
19. The method of any one of the paragraphs 6-18 and 130-134, wherein the method is performed in vitro.
20. The method of any one of the paragraphs 6-18 and 130-134, wherein the method is performed in vivo.
21. The method of any one of the paragraphs 6-20 and 130-134, wherein the mammalian cell is a human cell.
22. An engineered site-specific recombinase protein comprising a site-specific recombinase protein linked to protein tag capable of binding to a pore forming protein.
23. The engineered site-specific recombinase protein of paragraph 22, further comprising a nuclear localization signal.
24. The engineered site-specific recombinase protein of paragraphs 22 or 23, wherein the site-specific recombinase is a tyrosine recombinase or a serine recombinase.
25. The engineered site-specific recombinase protein of paragraph 24, wherein the tyrosine recombinase is Flp-recombinase or Cre-recombinase.
26. The engineered site-specific recombinase of paragraph 24, wherein the serine recombinase is PhiC31 Integrase.
27. The engineered site-specific recombinase protein of any one of paragraphs 22-26, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
28. A method for delivering an engineered site-specific recombinase into a mammalian cell comprising contacting the mammalian cell with a pore forming protein and the engineered site-specific recombinase of any one of the paragraphs 22-27.
29. The method of paragraph 28, wherein the pore forming protein further comprises a cell-targeting peptide.
30. The method of paragraph 29, wherein the cell-targeting peptide is selected from a cell receptor ligand, an antibody, or an affibody.
31. A method for site-specific modification of the genome of a mammalian cell comprising the steps of contacting the mammalian cell with a pore forming protein and the engineered site-specific recombinase of any one of the paragraphs 22-27.
32. The method of paragraph 31, wherein the core forming protein further comprises a cell targeting peptide.
33. The method of paragraph 32, wherein the cell-targeting peptide is selected from a cell receptor ligand, an antibody, or an affibody.
34. The method of paragraph 33, wherein the mammalian cell is contacted with at least two pairs of the engineered site-specific recombinases of any one of the paragraphs 22-27, wherein each of the at least two pairs of the engineered site-specific recombinases target a different receptor and are active only when transported via the different receptors into the same mammalian cell, thus minimizing both off-target activity and activity in non-target cells.
35. The method of paragraph 34, wherein the off-target activity comprised cutting DNA at a single recombinase binding site.
36. The method of paragraph 34, comprising contacting the cell with two site-specific recombinases, wherein the recombinases are selected from phage lambda integrase a Cre/loxP or a desired sequence-targeting engineered variant thereof and Flp/FRT or a desired sequence-targeted engineered variant thereof.
37. The method of paragraph 36, wherein the desired sequence-targeting engineered recombinases are TALE-recombinase and a catalytically dead Cas9 -recombinase.
38. The method of any one of paragraphs paragraph 28-37 and 135-138, further comprising a step of contacting the cell with a replacement nucleic acid.
39. The method of any one of the paragraphs 28-38 and 135-138, wherein the mammalian cell is a neuronal system cell.
40. The method of any one of the paragraphs 28-39 and 135-138, wherein the method is performed in vitro.
41. The method of any one of the paragraphs 28-39 and 135-138, wherein the method is performed in vivo.
42. The method of any one of the paragraphs 28-41 and 135-138, wherein the mammalian cell is a human cell.
43. An engineered site-specific enzyme protein comprising a site-specific enzyme protein linked to protein tag capable of binding to a pore forming protein.
44. The engineered site-specific enzyme protein of paragraph 43, further comprising a nuclear localization signal.
45. The engineered site-specific enzyme protein of paragraphs 43-44, wherein the site-specific enzyme protein is a LAGLIDADG homing endonuclease (“LAGLIDADG” disclosed as SEQ ID NO: 1).
46. The engineered site-specific enzyme protein of paragraphs 43-45, wherein the endonuclease is a monomeric endonuclease.
47. The engineered site-specific enzyme protein of any one of paragraphs 43-46, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
48. A method for delivering an engineered site-specific enzyme protein into a mammalian cell comprising contacting the mammalian cell with a pore forming protein and the engineered site-specific enzyme protein of any one of the paragraphs 43-47.
49. The method of paragraph 48, wherein the pore forming protein further comprises a cell-targeting peptide.
50. The method of paragraph 49, wherein the cell-targeting peptide is selected from a cell receptor ligand, an antibody, or an affibody.
51. A method for site-specific modification of the genome of a mammalian cell comprising the steps of contacting the mammalian cell with a pore forming protein and the engineered site-specific enzyme protein of any one of the paragraphs 43-50.
52. The method of paragraph 51, wherein the pore forming protein further comprises a cell targeting peptide.
53. The method of paragraph 52, wherein the cell-targeting peptide is selected from a cell receptor ligand, an antibody, or an affibody.
54. The method of paragraph 51-53, wherein the mammalian cell is contacted with at least two pairs of the engineered site-specific nucleases of any one of paragraphs 43-50, wherein each of the at least two pairs of the engineered site-specific nucleases target a different receptor and are active only when transported via the different receptors into the same mammalian cell, thus minimizing both off-target activity and activity in non-target cells.
55. The method of paragraph 54, wherein the off-target activity comprised cutting DNA at a single enzymatic cleavage site.
56. The method of any one of paragraphs paragraph 51-55 and 139-142, further comprising a step of contacting the cell with a replacement nucleic acid.
57. The method of any one of the paragraphs 51-56 and 139-142, wherein the mammalian cell is a neuronal system cell.
58. The method of any one of the paragraphs 51-57 and 139-142, wherein the method is performed in vitro.
59. The method of any one of the paragraphs 51-57 and 139-142, wherein the method is performed in vivo.
60. The method of any one of the paragraphs 51-59 and 139-142, wherein the mammalian cell is a human cell.
61. A pair of engineered proteins comprising:
   a) a first engineered protein comprising a first portion of a site-specific nuclease protein and a first protein tag capable of binding to a pore forming protein; and
   b) a second engineered protein comprising a second portion of a site-specific nuclease protein and a second protein tag capable of binding to a pore forming protein;
      wherein the first and second portions of the site-specific nuclease are not enzymatically active portions and wherein the first and second portions of the site-specific nuclease can form a enzymatically active complex.
62. The pair of engineered proteins of paragraph 61, further comprising a nuclear localization signal.
63. The pair of engineered proteins of paragraphs 61 or 62, wherein the site-specific nuclease is selected from a zinc-finger nuclease, a transcription activator-like effector nuclease, and a monomeric site-specific nuclease.
64. The pair of engineered proteins of paragraph 63, wherein the monomeric site-specific nuclease is an engineered GIY-YIG family protein.
65. The pair of engineered proteins of any of paragraphs 61-64, wherein the first and second protein tags are capable of binding to the same pore forming protein.
66. The pair of engineered proteins of paragraph 65, wherein the first and second protein tags are identical.
67. The pair of engineered proteins of any of paragraphs 61-64, wherein the first protein tag binds to a first pore forming protein and the second protein tag binds to a second pore forming protein.
68. The pair of engineered proteins of any one of paragraphs 61-67, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
69. A method for delivering an engineered site-specific nuclease into a mammalian cell comprising contacting the mammalian cell with a pore forming protein and the pair of engineered proteins of any one of the paragraphs 61-68.
70. The method of paragraph 69, wherein the pore forming protein further comprises a cell-targeting peptide.
71. The method of paragraph 70, wherein the cell-targeting peptide is selected from a cell receptor ligand, an antibody, or an affibody.
72. A method for site-specific modification of the genome of a mammalian cell comprising the steps of contacting the mammalian cell with a pore forming protein and the pair of engineered proteins of any one of the paragraphs 61-68.
73. The method of paragraph 72, wherein the core forming protein further comprises a cell targeting peptide.
74. The method of paragraph 73, wherein the cell-targeting peptide is selected from a cell receptor ligand, an antibody, or an affibody.
75. The method of paragraph 71, wherein the mammalian cell is contacted with at least two pairs of the pair of engineered proteins of any one of paragraphs 61-68, wherein each of the at least two pairs of the engineered site-specific nucleases target a different receptor and are active only when transported via the different receptors into the same mammalian cell, thus minimizing both off-target activity and activity in non-target cells.
76. The method of paragraph 75, wherein the off-target activity comprises cutting DNA at a single TALEN binding site.
77. The method of paragraph 75, wherein the at least two pairs of the engineered proteins comprise a TALEN and a CRISPR nuclease targeted to adjacent sequences in which FokI nuclease dimerization is required for cutting.
78. The method of paragraph 77, comprising use of a nicking version of the FokI nuclease in TALE or Cas9 fusions targeted to adjacent sequences, wherein the targeted nucleases bind and nick DNA separately, and a double strand break forms if the second cut was made before the first was repaired.
79. The method of paragraph 78 comprising protein complementation, wherein at least one of the nucleases is divided into two separate and inactive domains which are fused to TALE or Cas9 proteins, wherein DNA double strand breaks occur only when the fusions bind to their target sequences in the correct orientation.
80. The method of any one of paragraphs paragraph 69-80, further comprising a step of contacting the cell with a replacement nucleic acid.
81. The method of any one of the paragraphs 69-80, wherein the mammalian cell is a neuronal system cell.
82. The method of any one of the paragraphs 69-81, wherein the method is performed in vitro.
83. The method of any one of the paragraphs 69-81, wherein the method is performed in vivo.
84. The method of any one of the paragraphs 68-83, wherein the mammalian cell is a human cell.
85. A pair of engineered proteins comprising:
   c) a first engineered protein comprising a first portion of a site-specific recombinase protein and a first protein tag capable of binding to a pore forming protein; and
   d) a second engineered protein comprising a second portion of a site-specific recombinase protein and a second protein tag capable of binding to a pore forming protein;
      wherein the first and second portions of the site-specific recombinase are not enzymatically active portions and wherein the first and second portions of the site-specific recombinase can form a enzymatically active complex.
86. The pair of engineered proteins of paragraph 61, further comprising a nuclear localization signal.
87. The pair of engineered proteins of paragraph 85 or 86, wherein the site-specific recombinase is a tyrosine recombinase or a serine recombinase.
88. The pair of engineered proteins of paragraph 87, wherein the tyrosine recombinase is Flp-recombinase or Cre-recombinase.
89. The pair of engineered proteins of paragraph 87, wherein the serine recombinase is PhiC31 Integrase.
90. The pair of engineered proteins of any of paragraphs 85-89, wherein the first and second protein tags are capable of binding to the same pore forming protein.
91. The pair of engineered proteins of paragraph 90, wherein the first and second protein tags are identical.
92. The pair of engineered proteins of any of paragraphs 85-89, wherein the first protein tag binds to a first pore forming protein and the second protein tag binds to a second pore forming protein.
93. The pair of engineered proteins of any one of paragraphs 85-92, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
94. A method for delivering an engineered site-specific recombinase into a mammalian cell comprising contacting the mammalian cell with a pore forming protein and the pair of engineered proteins of any one of paragraphs 85-93.
95. The method of paragraph 94, wherein the pore forming protein further comprises a cell-targeting peptide.
96. The method of paragraph 94, wherein the cell-targeting peptide is selected from a cell receptor ligand, an antibody, or an affibody.
97. A method for site-specific modification of the genome of a mammalian cell comprising the steps of contacting the mammalian cell with a pore forming protein and the pair of engineered proteins of any one of paragraphs 85-93.
98. The method of paragraph 97, wherein the core forming protein further comprises a cell targeting peptide.
99. The method of paragraph 98, wherein the cell-targeting peptide is selected from a cell receptor ligand, an antibody, or an affibody.
100. The method of paragraph 99, wherein the mammalian cell is contacted with at least two pairs of the engineered proteins of any of paragraphs 85-93, wherein each of the at least two pairs of the engineered site-specific recombinases target a different receptor and are active only when transported via the different receptors into the same mammalian cell, thus minimizing both off-target activity and activity in non-target cells.
101. The method of paragraph 100, wherein the off-target activity comprised cutting DNA at a single recombinase binding site.
102. The method of paragraph 100, comprising contacting the cell with two site-specific recombinases, wherein the recombinases are selected from phage lambda integrase a Cre/loxP or a desired sequence-targeting engineered variant thereof and Flp/FRT or a desired sequence-targeted engineered variant thereof.
103. The method of paragraph 102, wherein the desired sequence-targeting engineered recombinases are TALE-recombinase and a catalytically dead Cas9 -recombinase.
104. The method of any one of paragraphs paragraph 94-103, further comprising a step of contacting the cell with a replacement nucleic acid.
105. The method of any one of the paragraphs 94-104, wherein the mammalian cell is a neuronal system cell.
106. The method of any one of the paragraphs 94-105, wherein the method is performed in vitro.
107. The method of any one of the paragraphs 94-105, wherein the method is performed in vivo.
108. The method of any one of the paragraphs 94-107, wherein the mammalian cell is a human cell.
109. A pair of engineered proteins comprising:
   e) a first engineered protein comprising a first portion of a site-specific enzyme protein and a first protein tag capable of binding to a pore forming protein; and
   f) a second engineered protein comprising a second portion of a site-specific enzyme protein and a second protein tag capable of binding to a pore forming protein;
      wherein the first and second portions of the site-specific enzyme are not enzymatically active portions and wherein the first and second portions of the site-specific enzyme can form a enzymatically active complex.
110. The pair of engineered proteins of paragraph 109, further comprising a nuclear localization signal.
111. The pair of engineered proteins of paragraphs 109 or 110, wherein the site-specific enzyme protein is a LAGLIDADG homing endonuclease (“LAGLIDADG” disclosed as SEQ ID NO: 1).
112. The pair of engineered proteins of paragraphs 109 or 110, wherein the endonuclease is a monomeric endonuclease.
113. The pair of engineered proteins of any of paragraphs 109-112, wherein the first and second protein tags are capable of binding to the same pore forming protein.
114. The pair of engineered proteins of paragraph 113, wherein the first and second protein tags are identical.
115. The pair of engineered proteins of any of paragraphs 109-112, wherein the first protein tag binds to a first pore forming protein and the second protein tag binds to a second pore forming protein.
116. The pair of engineered proteins of any one of paragraphs 109-115, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.
117. A method for delivering an engineered site-specific enzyme protein into a mammalian cell comprising contacting the mammalian cell with a pore forming protein and the pair of engineered proteins of any one of paragraphs 109-116.
118. The method of paragraph 117, wherein the pore forming protein further comprises a cell-targeting peptide.
119. The method of paragraph 118, wherein the cell-targeting peptide is selected from a cell receptor ligand, an antibody, or an affibody.
120. A method for site-specific modification of the genome of a mammalian cell comprising the steps of contacting the mammalian cell with a pore forming protein and the pair of engineered proteins of any one of paragraphs 109-116.
121. The method of paragraph 120, wherein the pore forming protein further comprises a cell targeting peptide.
122. The method of paragraph 121, wherein the cell-targeting peptide is selected from a cell receptor ligand, an antibody, or an affibody.
123. The method of paragraph 120-122, wherein the mammalian cell is contacted with at least two pairs of the pair of engineered proteins of any one of paragraphs 109-116, wherein each of the at least two pairs of the engineered proteins target a different receptor and are active only when transported via the different receptors into the same mammalian cell, thus minimizing both off-target activity and activity in non-target cells.
124. The method of paragraph 123, wherein the off-target activity comprised cutting DNA at a single enzymatic cleavage site.
125. The method of any one of paragraphs paragraph 120-124, further comprising a step of contacting the cell with a replacement nucleic acid.
126. The method of any one of the paragraphs 120-125, wherein the mammalian cell is a neuronal system cell.
127. The method of any one of the paragraphs 120-126, wherein the method is performed in vitro.
128. The method of any one of the paragraphs 120-126, wherein the method is performed in vivo.
129. The method of any one of the paragraphs 120-128, wherein the mammalian cell is a human cell.
130. The method of paragraph 8, wherein the mammalian cell is contacted with at least one pair of the engineered site-specific nucleases of any one of paragraphs 1-5, wherein each of the engineered site-specific nucleases targets a different receptor, and is active only when both of the engineered site-specific nucleases of the pair are transported via the different receptors into the same mammalian cell, thus minimizing both off-target activity and activity in non-target cells.
131. The method of paragraph 130, wherein the off-target activity comprises cutting DNA at a single TALEN binding site.
132. The method of paragraph 130, wherein the at least two pairs of the engineered site specific nucleases comprise a TALEN and a CRISPR nuclease targeted to adjacent sequences in which FokI nuclease dimerization is required for cutting.
133. The method of paragraph 130, comprising use of a nicking version of the FokI nuclease in TALE or Cas9 fusions targeted to adjacent sequences, wherein the targeted nucleases bind and nick DNA separately, and a double strand break forms if the second cut was made before the first was repaired.
134. The method of paragraph 132, comprising protein complementation, wherein at least one of the nucleases is divided into two separate and inactive domains which are fused to TALE or Cas9 proteins, wherein DNA double strand breaks occur only when the fusions bind to their target sequences in the correct orientation.
135. The method of paragraph 33, wherein the mammalian cell is contacted with at least one pair of the engineered site-specific recombinases of any one of the paragraphs 22-27, wherein each of the engineered site-specific recombinases targets a different receptor, and is active only when both of the engineered site-specific recombinases of the pair are transported via the different receptors into the same mammalian cell, thus minimizing both off-target activity and activity in non-target cells.
136. The method of paragraph 135, wherein the off-target activity comprised cutting DNA at a single recombinase binding site.
137. The method of paragraph 135, comprising contacting the cell with two site-specific recombinases, wherein the recombinases are selected from phage lambda integrase a Cre/loxP or a desired sequence-targeting engineered variant thereof and Flp/FRT or a desired sequence-targeted engineered variant thereof.
138. The method of paragraph 137, wherein the desired sequence-targeting engineered recombinases are TALE-recombinase and a catalytically dead Cas9 -recombinase.
139. The method of any of paragraphs 51-53, wherein the mammalian cell is contacted with at least one pair of the engineered site-specific enzymes of any one of paragraphs 43-50, wherein each of the engineered site-specific enzymes target a different receptor, and is active only when both the engineered site-specific enzymes are transported via the different receptors into the same mammalian cell, thus minimizing both off-target activity and activity in non-target cells.
140. The method of paragraph 139, wherein the off-target activity comprised cutting DNA at a single enzymatic cleavage site.
141. The method of any of paragraphs 51-53, wherein the mammalian cell is contacted with at least two pairs of the engineered site-specific enzyme proteins of any one of paragraphs 43-50, wherein each of the at least two pairs of the engineered site-specific enzyme proteins target a different receptor and are active only when transported via the different receptors into the same mammalian cell, thus minimizing both off-target activity and activity in non-target cells.
142. The method of paragraph 141, wherein the off-target activity comprised cutting DNA at a single enzymatic cleavage site.
143. The composition or method of any of paragraphs 1-142, wherein the protein tag comprises a portion of anthrax lethal factor.
144. The composition or method of paragraph 143, wherein the portion of anthrax lethal factor comprises LFn (at least residues 1-254 of anthrax lethal factor).
145. A nucleic acid encoding the polypeptide composition of any one of paragraphs 1-144.

### EXAMPLES

### Example 1

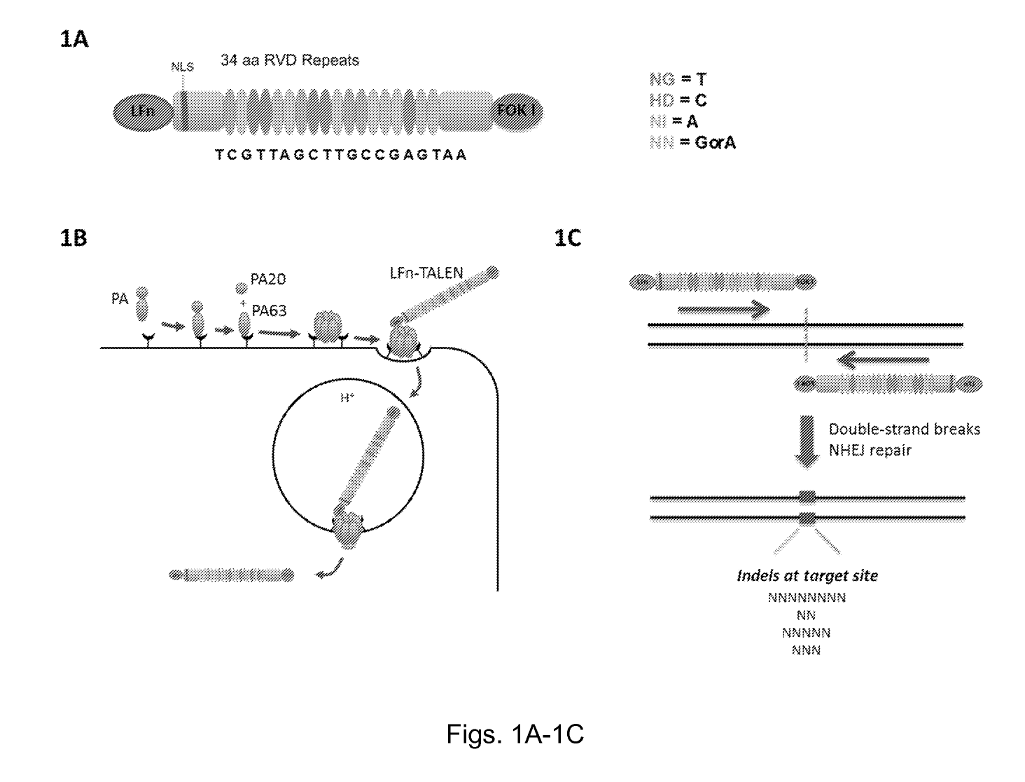

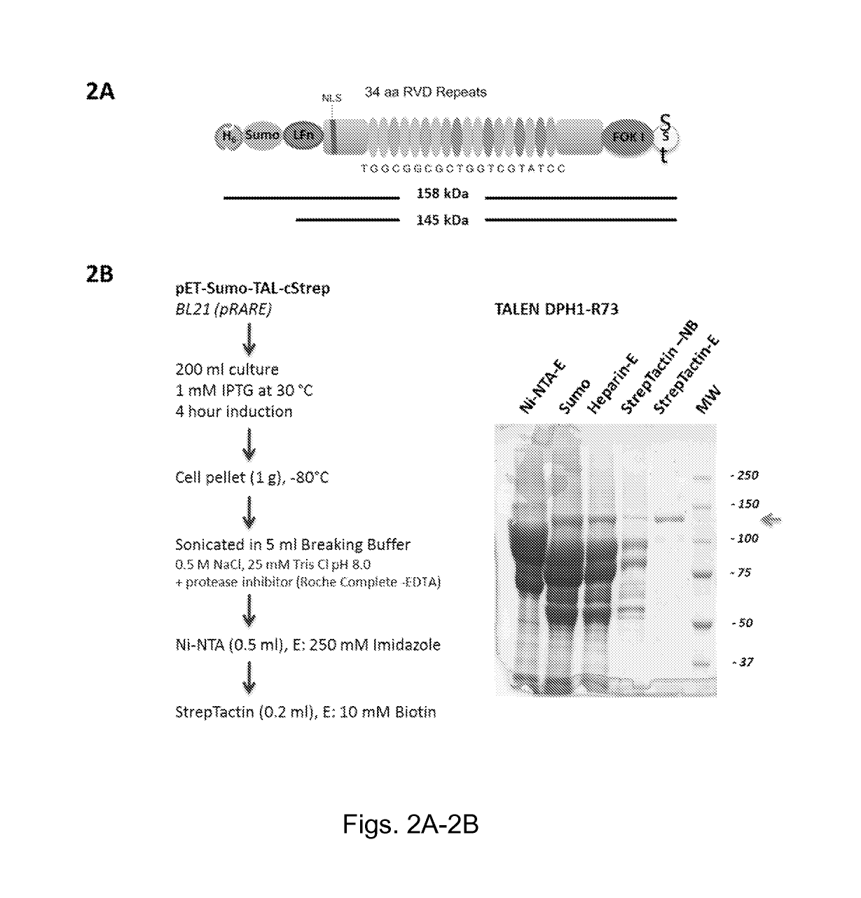

We purified LFn-tagged TALEN proteins into cells via anthrax protective antigen (PA) pores. FIGS. 1A-1C show the method, where we introduced purified LFn-tagged TALEN proteins into cells via protective antigen pores to allow specific cuts at a genomic DNA target sequence followed by non-homologous end-joining DNA repair and loss of gene function though reading frame alterations.

### Example 2

We showed that the TALEN proteins entered the nucleus and maintained their functionality. FIGS. 2A-2C demonstrate that both purified and partially purified LFn-TALEN proteins have site-specific endonuclease activity in vitro. FIG. 3 demonstrates that both purified and partially purified LFn-TALEN proteins can induce loss of function in a counter-selectable gene (DPH1) when added to human cells along with protective antigen.

### Example 3

Recombinase-mediated cassette exchange (RMCE) is a useful technique for introducing multiple alleles or markers efficiently into a defined site in the genome. It is accomplished by surrounding the cassette DNA to be exchanged with recombination sites (e.g., loxP) in both the targeting and the genomic target sequences in cells expressing the relevant recombinase (e.g., Cre).

A version of RMCE has been developed that utilizes two recombinase systems, Cre/loxP and Flp/FRT, to mediate the fragment exchange in way that mitigates the reversibility inherent to these recombination systems (Lauth et al., Nucleic Acids Research, 2002, Vol. 30 No. 21 e115). In this version of RMCE, the cassette DNA in both the donor (targeting) and genomic sites is flanked by LoxP and FRT sites (i.e., as loxP-cassette-FRT), and the recombination takes place in cells expressing both the Flp and Cre recombinases. An improved version of the method utilizes balanced co-expression of Cre and Flp recombinases, that are expressed from a single promoter as Flp-2A-Cre or Flp-IRES-Cre, and yields a high fraction of cells with fragment replacement (Anderson et al., Nucleic Acids Research, 2012, Vol. 40, No. 8 e62).

One can also solve the reversibility using another common approach to the Cre and Flp recombinase, namely using variant recombination sites at either end of the DNA to be exchanged (e.g., the loxP site variants used in CREATOR™ (Clontech) or Univector cloning (see, e.g. Liu et al. Curr Biol. 1998 Dec. 3; 8(24):1300-9) and the att site variants used in GATEWAY® cloning (Invitrogen).

One can also use RMCE using dual transposon integrases (e.g., piggybac or Sleeping Beauty), or for a dual integrase (PhiC31 Integrase and a partner) version of integrase-mediated site-specific insertion (IMSI).

### Example 4

### Translocation of ZFN's using Anthrax Platform

Described herein is the delivery of ZFNs into cells using the Anthrax Toxin platform to achieve site specific genome editing. Delivery of a single zinc finger, Fold, a NLS-tagged LFN, and a NLS-ZFN is described.

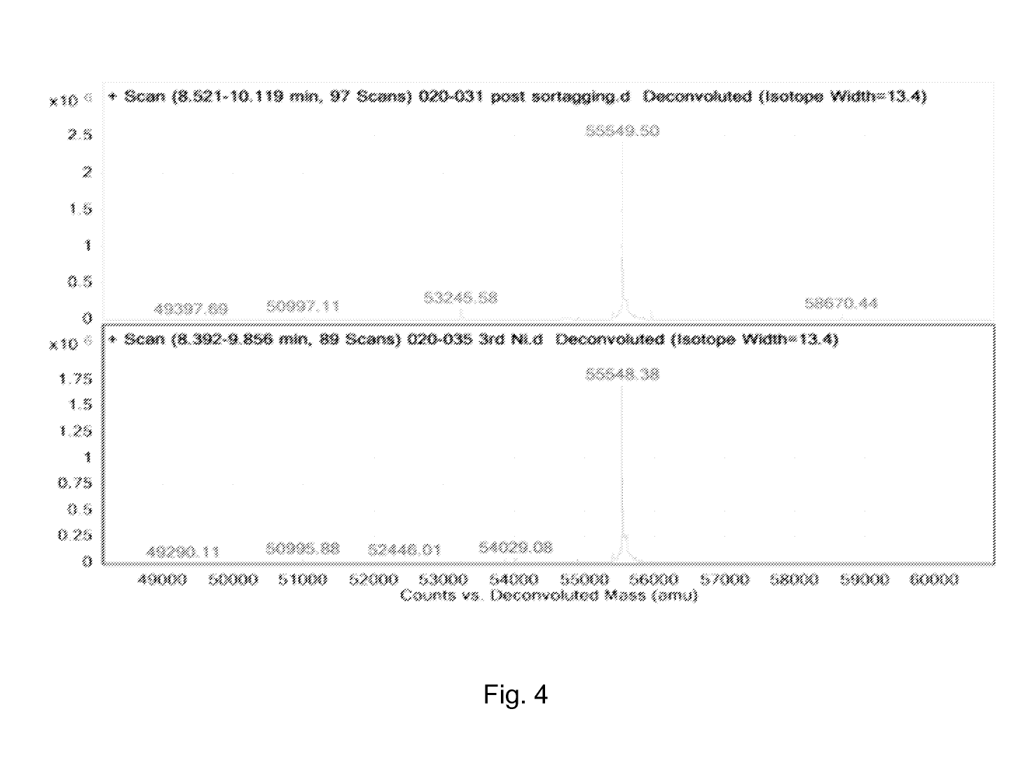

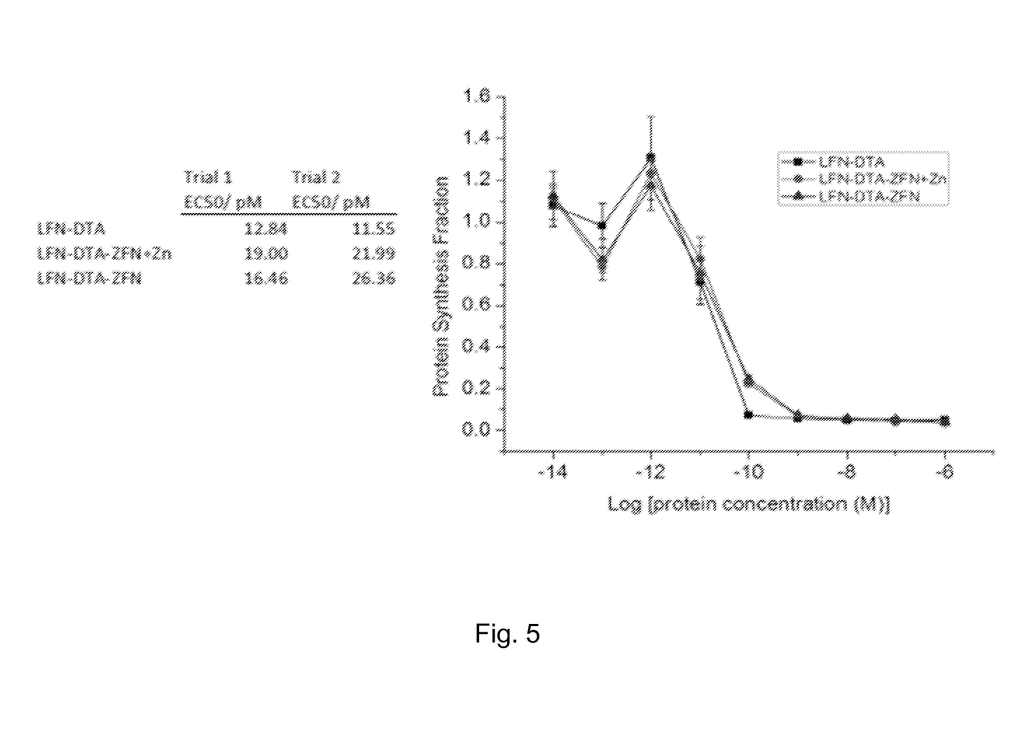

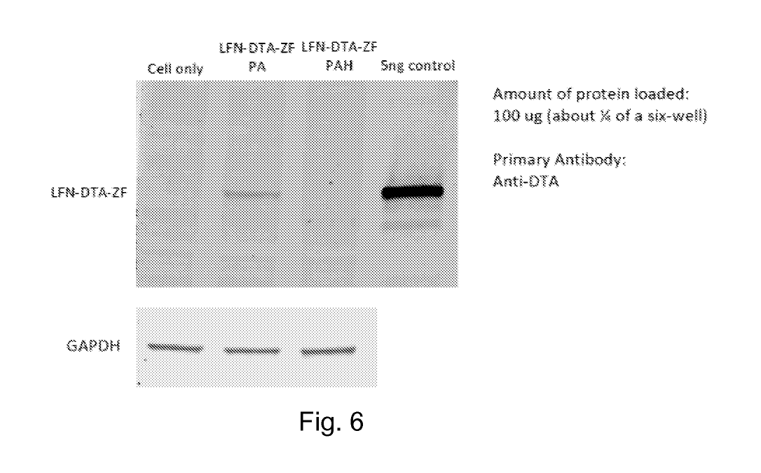

Delivery of LFN-DTA-ZFN is demonstrated in FIGS. 4, 5, and 6. FIG. 4 shows the LCMS trace of purified single zinc finger fused to LFN-DTA by sortagging. FIG. 5 demonstrates LFN-DTA-ZF can be delivered as effectively as the positive control LFN-DTA by protein synthesis inhibition assay. FIG. 6 shows the delivery of LFN-DTA-ZF by western blot. The LFN-DTA-ZF band can only be observed in the absence of wild type PA. In the presence of PA(F427H), a PA mutant that abolishes its translocation ability, no band can be observed.

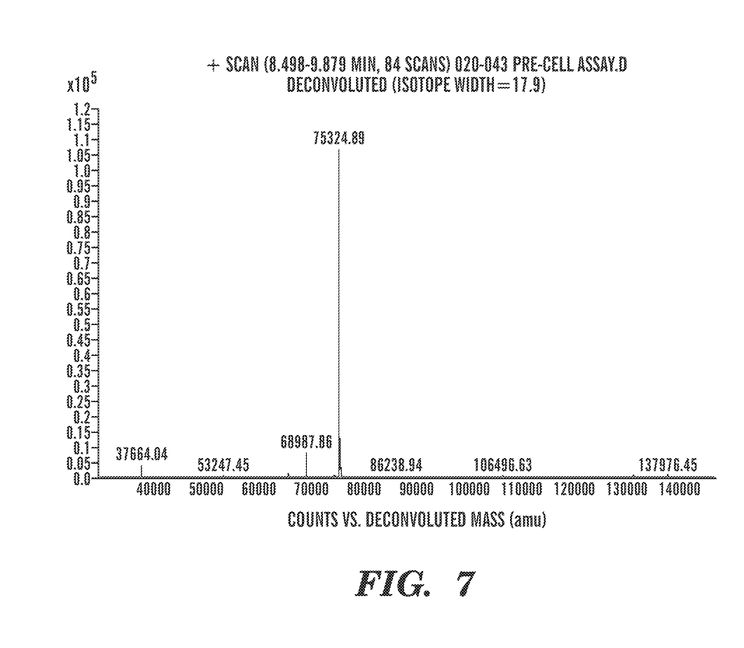

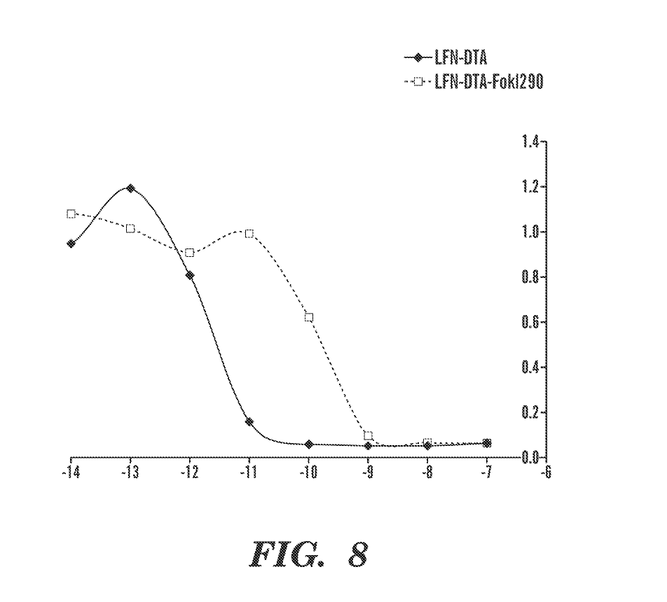

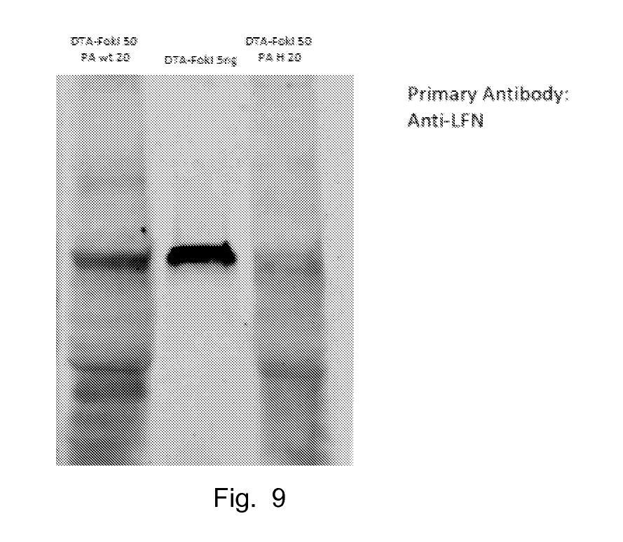

Delivery of LFN-DTA-FokI is demonstrated in FIGS. 7, 8, and 9. FIG. 7 shows the LCMS trace of purified LFN-DTA-FokI. FIG. 8 shows LFN-DTA-FokI has a two-log shift compared to LFN-DTA, indicating translocation is affected by FokI most likely due to the presence of cysteine. FIG. 9 demonstrates the translocation of LFN-DTA-FokI by western blot in HEK293T cells. The LFN-DTA-FokI band can still be seen in the presence of 200 nM of LFN-DTA-FokI and 40 nM of wild-type PA after 24 hours of incubation despite its affected translocation efficiency, implying the some amount of LFN-DTA-FokI can still be delivered into cells by PA.

NLS tagged LFN, tagged with either HA tag or Flag tag was incubated with CHO cells for 1 or 4 hours and incubated with HEK cells overnight to demonstrate delivery (data not shown).

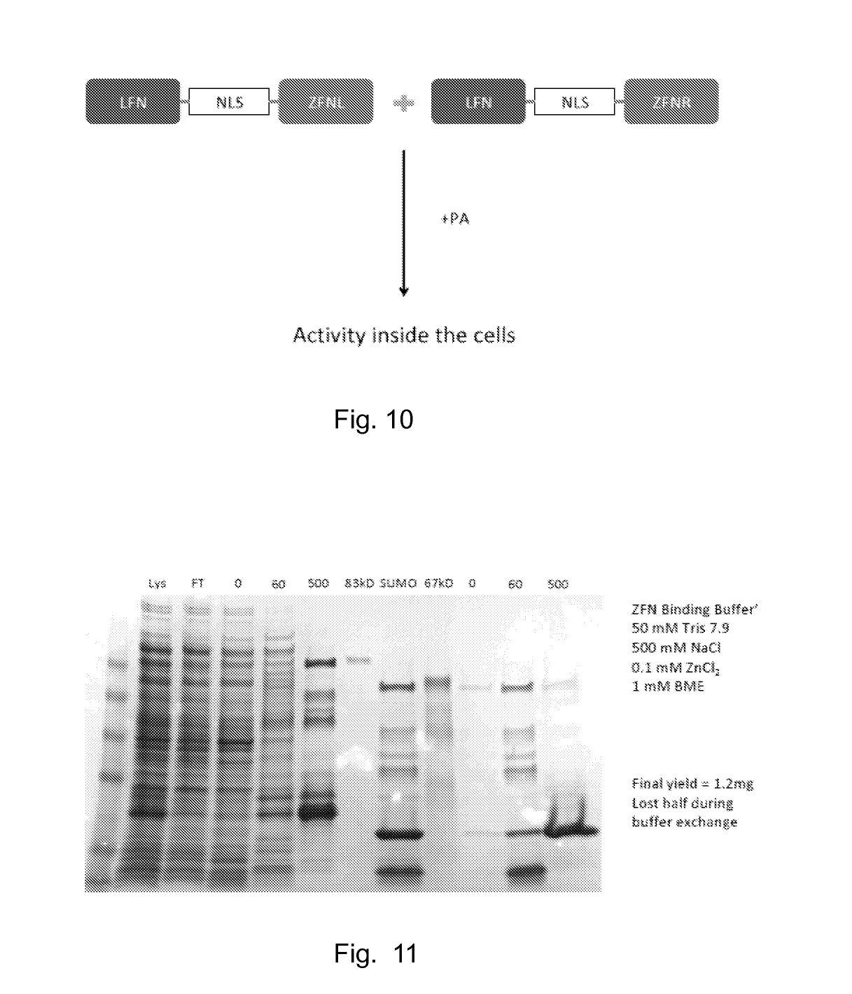

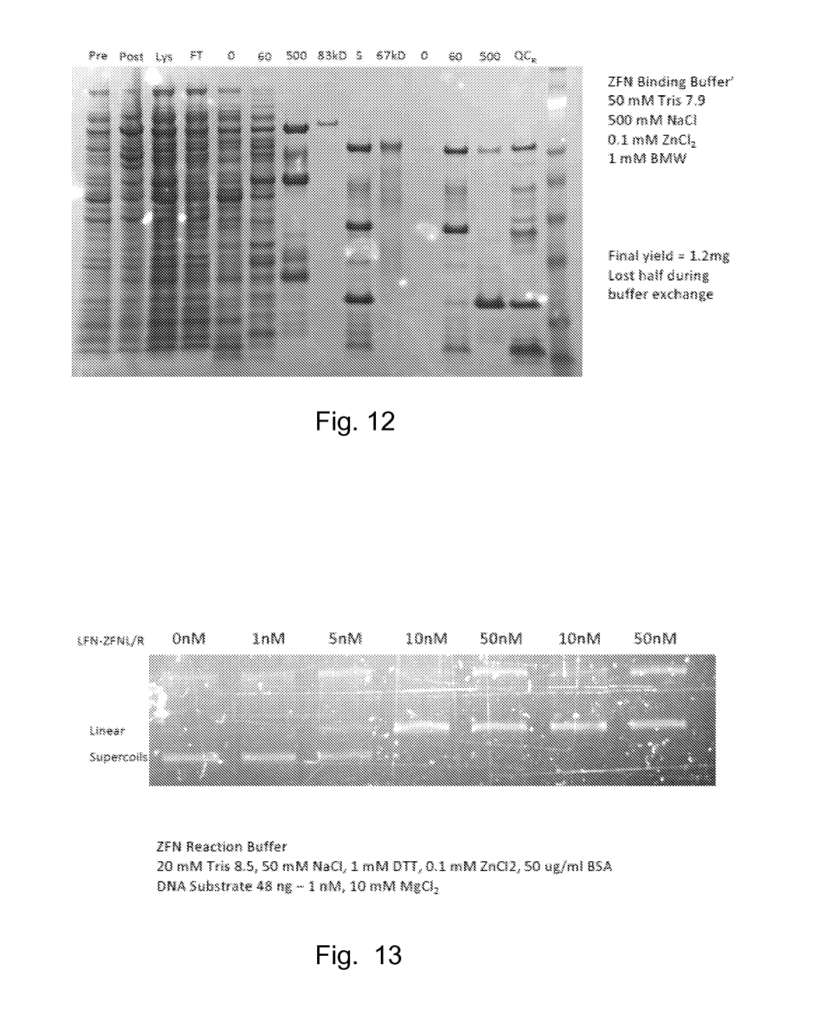

Delivery of LFN-ZFN was accomplished according to the strategy depicted in FIG. 10. LFN-ZFNR and LFN-ZFNL were purified by Ni columns separately (FIGS. 11 and 12 respectively) and an in vitro cleavage assay was performed using the combination of LFN-ZFNR and LFN-ZRNL (FIG. 13) to demonstrate that the combination of proteins has cleavage activity, indicated by upward shifts of the bands.

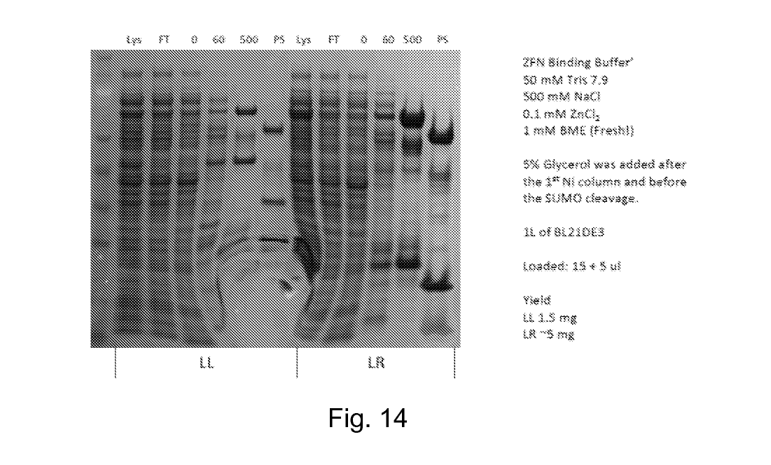

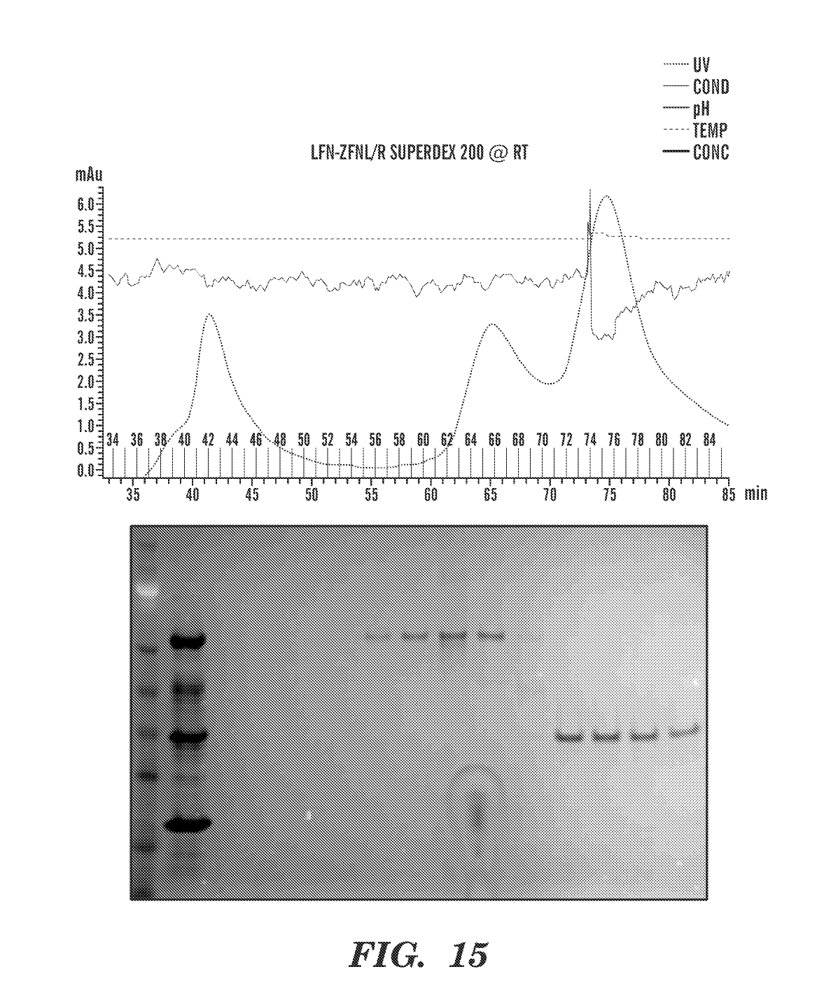

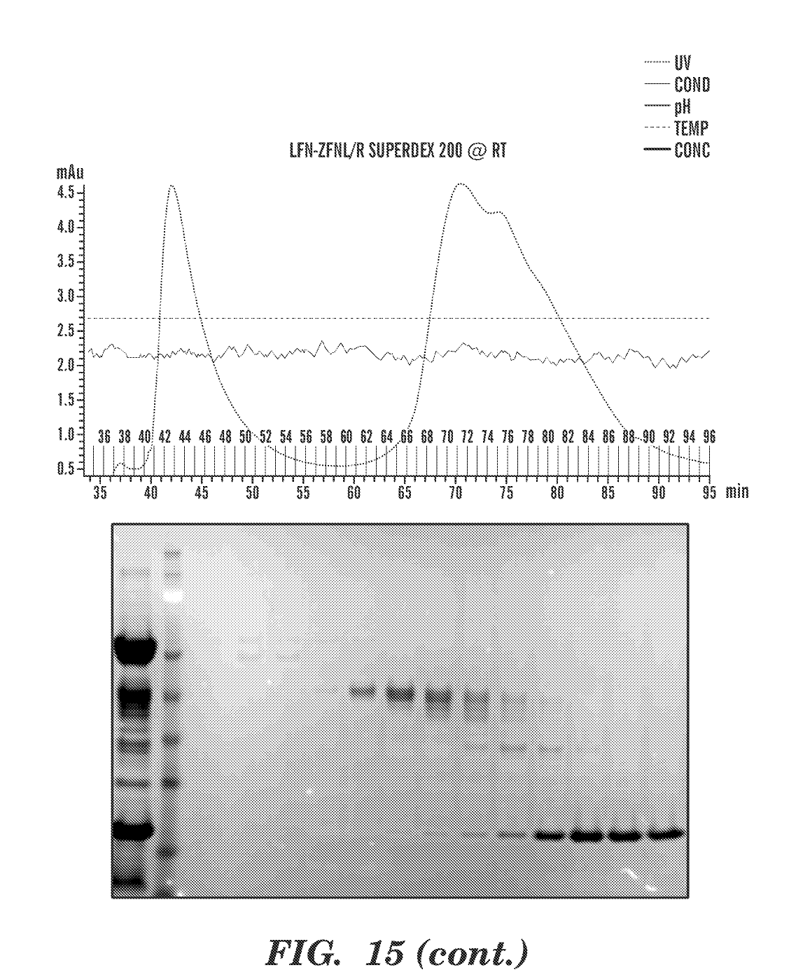

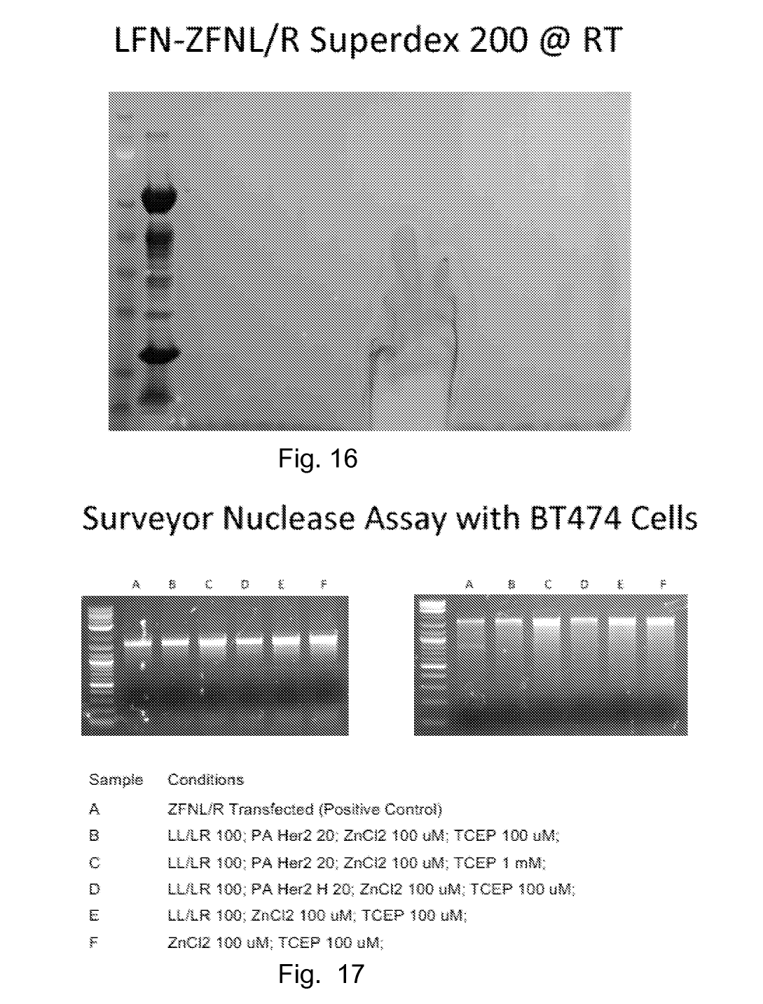

Some additional figures of purification of LFN-ZFNR/L are shown in FIGS. 14-16.

FIG. 17 shows the activity of LFN-ZFNR/L in BT474 cells measured by surveyor nuclease assays. BT474 cells were incubated with 100 nM of LFN-ZFNR/L in the presence of 20 nM of PA-Her2 or PA(F427H)-Her2. BT474 cells transfected with ZFNR/L served as positive control. Surveyor nuclease is known to cleave mismatched region. Therefore, the band below the major band indicates the cleavage of the target site. The first gel is the amplification step of the surveyor nuclease assay and no other band except the major band is supposed to be observed at this step.

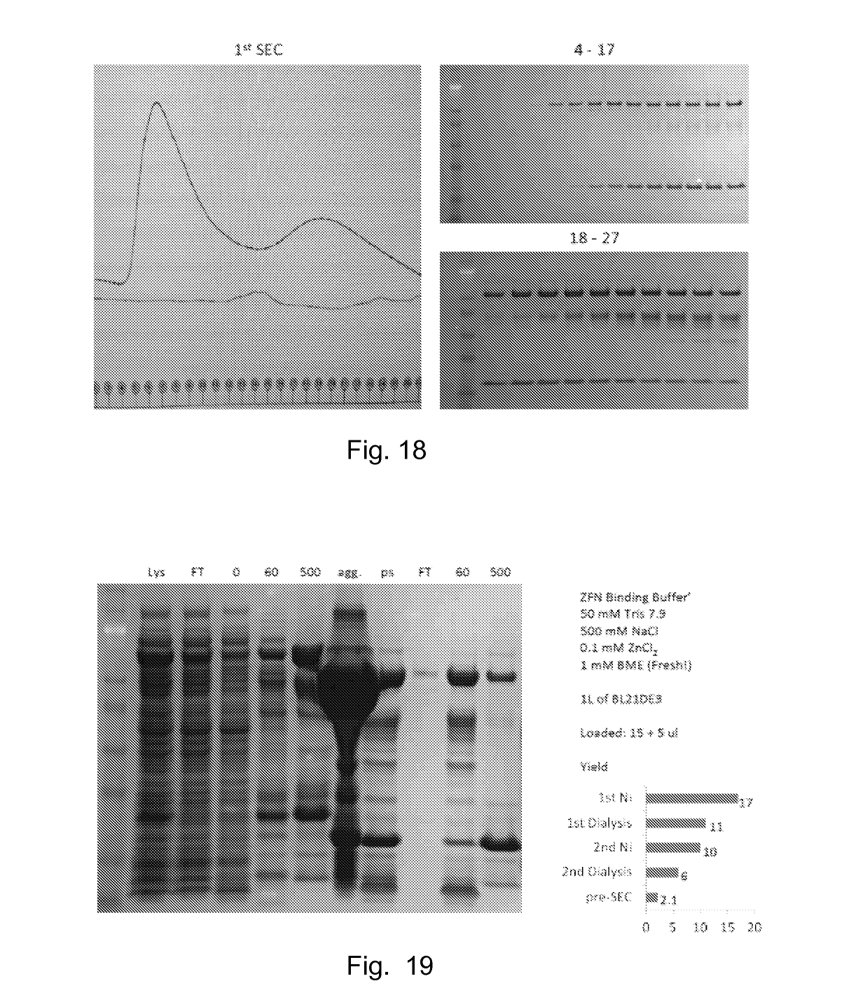

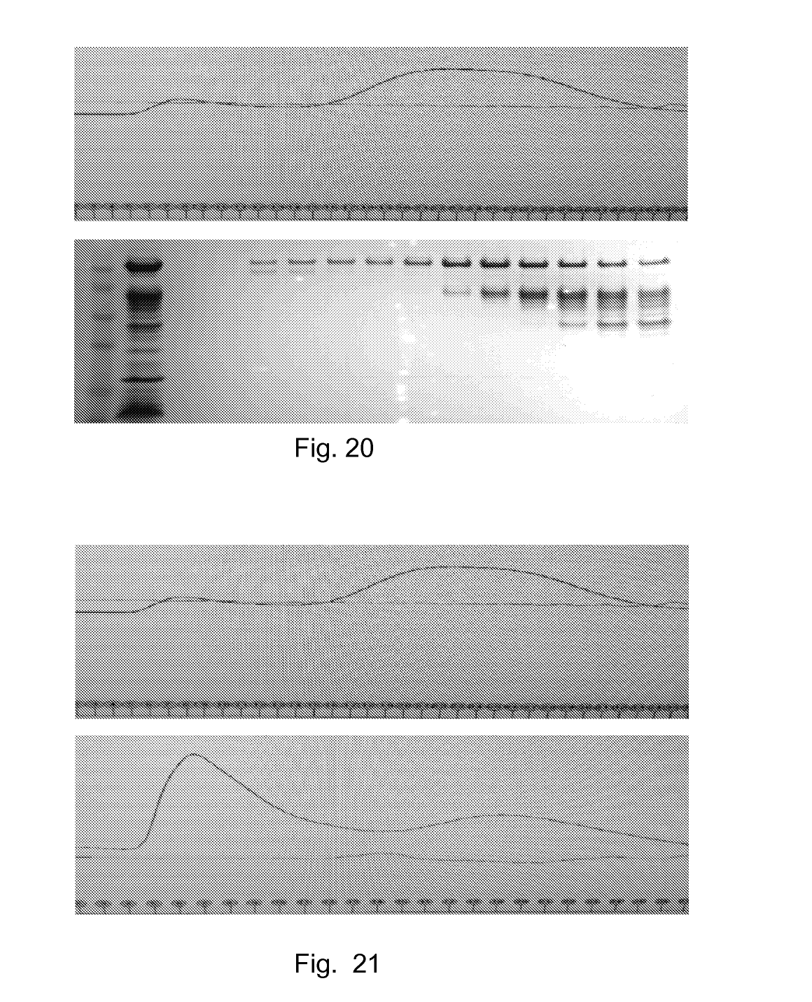

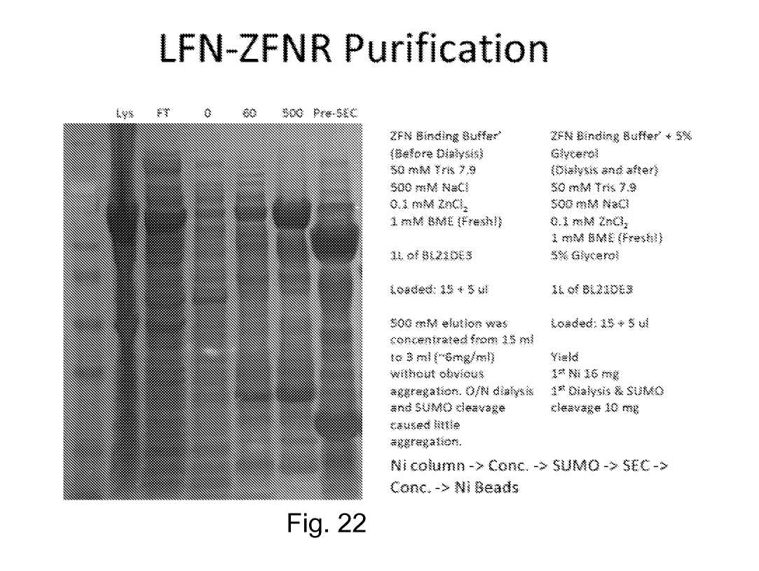

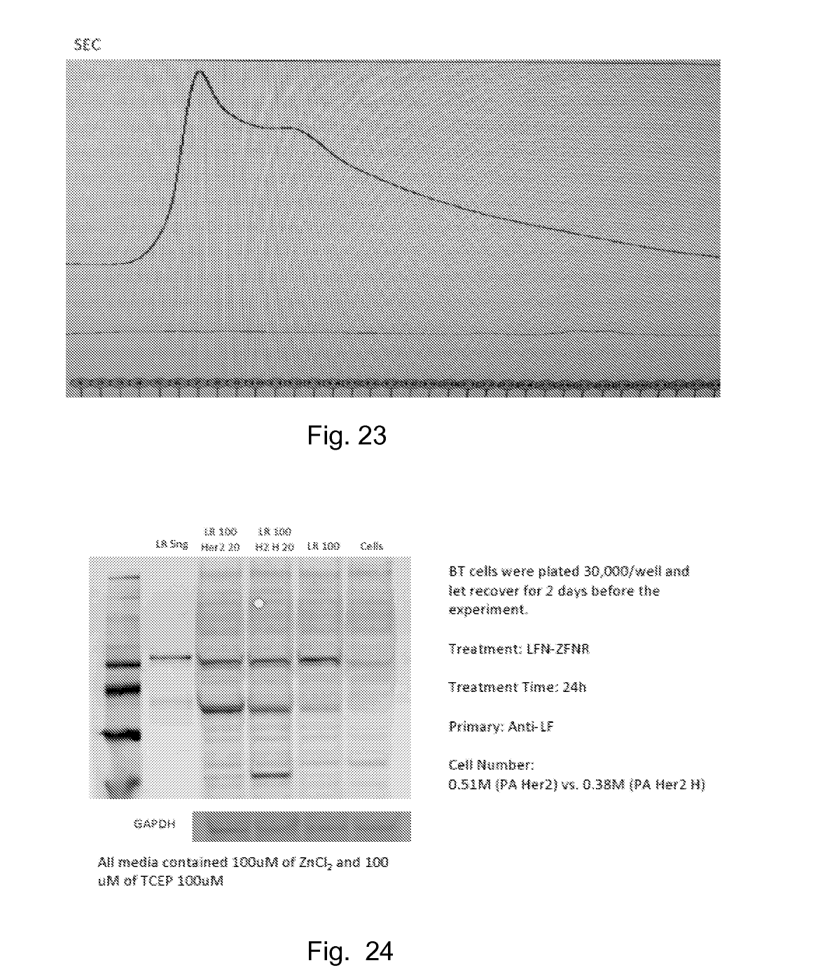

The optimization of purification of LFN-ZFNR is demonstrated in FIGS. 18-23.

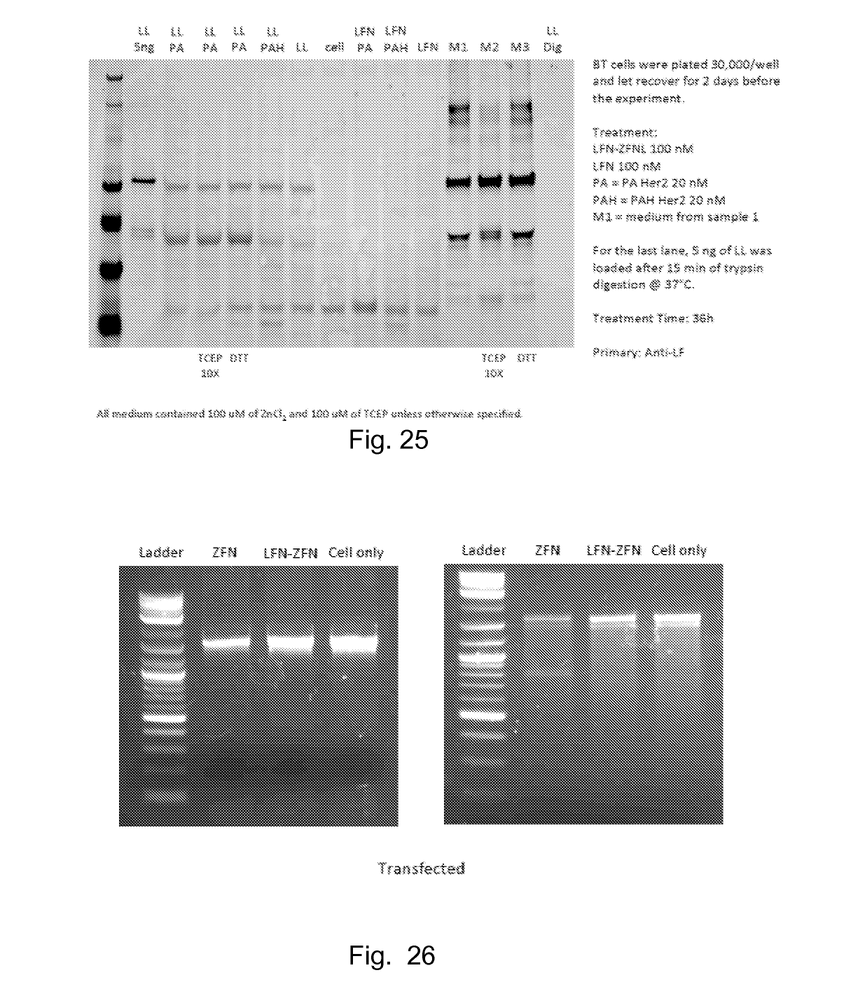

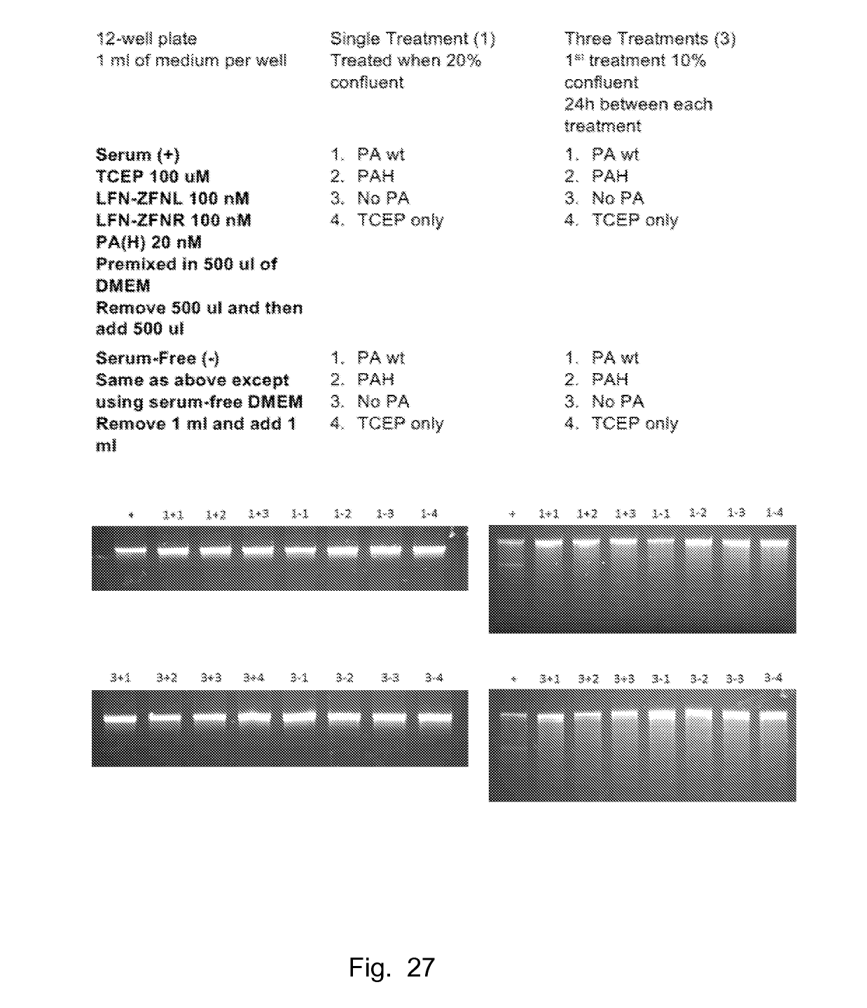

FIGS. 24-25 depict western blots of BT474 cells treated with LFN-ZFNR (FIG. 24) and LFN-ZFNL (FIG. 25). BT474 cells were incubated with LFN-ZFNR or LFN-ZFNL in the presence of PA-Her2 or PA(F427H)-Her2 for 24 or 36 hours. The major band in the 5 ng loading lane indicates the molecular weight of the cargo protein. As seen in the figures, both LFN-ZFNR and LFN-ZFNL are prone to cleavage. The cleavage is exacerbated in the medium. As a result of this, both the full length and the truncated bands are present in the treatment samples. 100 uM of ZnCl₂ was added to keep ZFN folded and TCEP was used as a reducing reagent. FIG. 26 depicts the results of a surveyor nuclease assay for HEK cells transfected with ZFN or LFN-ZFN. It was observed that the transfection of LFN-ZFN failed to cause any activity inside HEK cells. However, this result was most likely due to the failure of overexpression of LFN-ZFN after transfection (data not shown). FIG. 27 shows the effect of multiple treatments and serum. BT474 cells were subjected to single or three treatments of LFNR/L and PA in the presence or absence of serum.

1. An engineered site-specific nuclease protein comprising a site-specific nuclease protein and a protein tag capable of binding to a pore forming protein.

2. The engineered site-specific nuclease protein of claim 1, further comprising a nuclear localization signal.

3. The engineered site-specific nuclease protein of claim 1, wherein the site-specific nuclease is selected from a zinc-finger nuclease, a transcription activator-like effector nuclease, and a monomeric site-specific nuclease.

4. The engineered site-specific nuclease protein of claim 3, wherein the monomeric site-specific nuclease is an engineered GIY-YIG family protein.

5. The engineered site-specific nuclease protein of claim 1, wherein the pore forming protein is selected from Cholera toxin; Diphtheria toxin; Shiga toxin; Shiga-like toxins 1 and 2; Pseudomonas exotoxins (A, U, S, T, Y); Heat-labile enterotoxin; Anthrax toxin (binary); Adenylyl cyclase toxin; Tetanus toxin; Clostridium Botulinum neurotoxins (A, B, C, D, E, F, G); B. fragilis toxin; Dermonecrotic toxin; Cytotoxic necrotic factor 1 and 2; Cytolethal distending toxins; Clostridium perfringens toxins (alpha, beta, epsilon, iota) (binary); Clostridium spiroforme Iota-like toxins (binary); Clostridium difficile toxins A and B (binary); Clostridium difficile toxins A and B (single-chain); Clostridium C2 toxin (binary); Clostridium sordelli lethal toxin; Pertussis toxin; Ricin; Intermedilysin; Streptolysin O; Aerolysin; Staphylococcus alpha toxin; Alpha-hemolysin; Vibrio MARTX toxins; Pasteurella multicida toxin; Clostridium Botulinum Exoenzyme C3; Clostridium limosum C3-like toxins; Clostridium novyi alpha-toxin; Staphylococcus aureus EDIN toxin; Staphylococcus aureus C3stau toxin; Saporin; Trichosanthin; Abrin; Gelonin; pokeweed antiviral protein; pepcin; maize RIP; alpha-sarcin.

6. A method for delivering an engineered site-specific nuclease into a mammalian cell comprising contacting the mammalian cell with a pore forming protein and the engineered site-specific nuclease selected from the group of engineered site-specific nuclease proteins of any one of the claims 1-5.

7. The method of claim 6, wherein the pore forming protein further comprises a cell-targeting peptide.

8. The method of claim 7, wherein the cell-targeting peptide is selected from a cell receptor ligand, an antibody, or an affibody.

9. A method for site-specific modification of the genome of a mammalian cell comprising the steps of contacting the mammalian cell with a pore forming protein and the engineered site-specific nuclease selected from the group of engineered site-specific nuclease proteins of any one of the claims 1-5.

10. The method of claim 9, wherein the core forming protein further comprises a cell targeting peptide.

11. The method of claim 10, wherein the cell-targeting peptide is selected from a cell receptor ligand, an antibody, or an affibody.

12. The method of claim 8, wherein the mammalian cell is contacted with at least two pairs of the engineered site-specific nucleases selected from the group of engineered site-specific nuclease proteins of any one of claims 1-5, wherein each of the at least two pairs of the engineered site-specific nucleases target a different receptor and are active only when transported via the different receptors into the same mammalian cell, thus minimizing both off-target activity and activity in non-target cells.

13. The method of claim 12, wherein the off-target activity comprises cutting DNA at a single TALEN binding site.

14. The method of claim 12, wherein the at least two pairs of the engineered site specific nucleases comprise a TALEN and a CRISPR nuclease targeted to adjacent sequences in which FokI nuclease dimerization is required for cutting.

15. The method of claim 12, comprising use of a nicking version of the FokI nuclease in TALE or Cas9 fusions targeted to adjacent sequences, wherein the targeted nucleases bind and nick DNA separately, and a double strand break forms if the second cut was made before the first was repaired.

16. The method of claim 14, comprising protein complementation, wherein at least one of the nucleases is divided into two separate and inactive domains which are fused to TALE or Cas9 proteins, wherein DNA double strand breaks occur only when the fusions bind to their target sequences in the correct orientation.

17. The method of any one of claim 9, further comprising a step of contacting the cell with a replacement nucleic acid.

18-21. (canceled)

22. An engineered site-specific recombinase protein comprising a site-specific recombinase protein linked to protein tag capable of binding to a pore forming protein.

23-60. (canceled)

61. A pair of engineered proteins comprising:
a) a first engineered protein comprising a first portion of a site-specific nuclease protein and a first protein tag capable of binding to a pore forming protein; and
b) a second engineered protein comprising a second portion of a site-specific nuclease protein and a second protein tag capable of binding to a pore forming protein;
wherein the first and second portions of the site-specific nuclease are not enzymatically active portions and wherein the first and second portions of the site-specific nuclease can form a enzymatically active complex.

62-84. (canceled)

85. A pair of engineered proteins comprising:
c) a first engineered protein comprising a first portion of a site-specific recombinase protein and a first protein tag capable of binding to a pore forming protein; and
d) a second engineered protein comprising a second portion of a site-specific recombinase protein and a second protein tag capable of binding to a pore forming protein;
wherein the first and second portions of the site-specific recombinase are not enzymatically active portions and wherein the first and second portions of the site-specific recombinase can form a enzymatically active complex.

86-143. (canceled)


SEQUENCE LISTING (8 sequences)

SEQ ID NO: 1 - protein, 9 aa, Artificial Sequence
  Description of Artificial Sequence Synthetic peptide
LAGLIDADG

SEQ ID NO: 2 - protein, 25 aa, Artificial Sequence
  Description of Artificial Sequence Synthetic peptide
MLSLRQSIRFFKPATRTLCSSRYLL

SEQ ID NO: 3 - protein, 8 aa, Artificial Sequence
  Description of Artificial Sequence Synthetic peptide
PPKKKRKV

SEQ ID NO: 4 - protein, 10 aa, Artificial Sequence
  Description of Artificial Sequence Synthetic peptide
LADGLIDADG

SEQ ID NO: 5 - protein, 58 aa, Artificial Sequence
  Description of Artificial Sequence Synthetic polypeptide
VDNKFDKEXXXAXXEIXXLPNLNXXQXXAFIXSLXDDPSQSADLLAEAKKLDDAQAPK

SEQ ID NO: 6 - DNA, 19 bp, Artificial Sequence
  Description of Artificial Sequence Synthetic oligonucleotide
tcgttagcttgccgagtaa

SEQ ID NO: 7 - protein, 6 aa, Artificial Sequence
  Description of Artificial Sequence Synthetic 6xHis tag
HHHHHH

SEQ ID NO: 8 - DNA, 20 bp, Artificial Sequence
  Description of Artificial Sequence Synthetic oligonucleotide
tggcggcgctggtcgtatcc